(19) United States
(12) Patent Application Publication
(10) Pub. No.: US 2025/0066322 A1
(43) Pub. Date: Feb. 27, 2025

(54) SUBSTITUTED HETEROCYCLIC COMPOUNDS

(71) Applicant: BRISTOL-MYERS SQUIBB COMPANY, PRINCETON, NJ (US)

(72) Inventors: MICHAEL EDWARD MERTZMAN, NEW HOPE, PA (US); RYAN M. MOSLIN, PRINCETON, NJ (US); STEVEN H. SPERGEL, WARRINGTON, PA (US); KARIN IRMGARD WORM, JACKSON, NJ (US)

(73) Assignee: BRISTOL-MYERS SQUIBB COMPANY, PRINCETON, NJ (US)

(21) Appl. No.: 18/560,520

(22) PCT Filed: May 13, 2022
(86) PCT No.: PCT/US2022/029112

§ 371 Date: Nov. 13, 2023

Related U.S. Application Data
(60) Provisional application No. 63/188,498, filed on May 14, 2021.
(60) Provisional application No. 63/340,629, filed on May 11, 2022.

(51) Int. Cl. C07D 401/12; A61K 31/444; A61K 31/501

(52) U.S. Cl. CPC C07D 401/12; A61K 31/444; A61K 31/501

(57) ABSTRACT

There are disclosed compounds of the following formula (I): or a stereoisomer or pharmaceutically acceptable salt thereof, wherein all substituents are as defined herein, which are useful in the modulation of IL-12, IL-23 and/or IFNα, by acting on Tyk-2 to cause signal transduction inhibition. The compounds of the invention may be useful for treating neurodegenerative diseases or disorders.

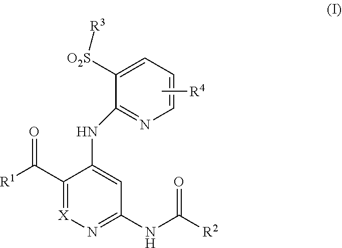

### CROSS-REFERENCE TO RELATED APPLICATIONS

This application claims the benefit of U.S. Provisional Application No. 63/188,498, filed May 14, 2021, and U.S. Provisional Application No. 63/340,629, filed May 11, 2022, the disclosures of which are incorporated herein by reference in its entirety.

### FIELD OF THE INVENTION

This invention relates to compounds useful in the modulation of IL-12, IL-23 and/or IFNα by acting on Tyk-2 to cause signal transduction inhibition. Provided herein are -substituted heterocyclic compounds, compositions comprising such compounds, and methods of their use. The invention further pertains to pharmaceutical compositions containing at least one compound according to the invention that are useful for the treatment of conditions related to the modulation of IL-12, IL-23 and/or IFNα in a mammal. In particular, this invention relates to compounds which show utility against neurodegenerative diseases.

### BACKGROUND OF THE INVENTION

The heterodimeric cytokines interleukin (IL)-12 and IL-23, which share a common p40 subunit, are produced by activated antigen-presenting cells and are critical in the differentiation and proliferation of Th1 and Th17 cells, two effector T cell lineages which play key roles in autoimmunity. IL-23 is composed of the p40 subunit along with a unique p19 subunit. IL-23, acting through a heterodimeric receptor composed of IL-23R and IL-12Rβ1, is essential for the survival and expansion of Th17 cells which produce pro-inflammatory cytokines such as IL-17A, IL-17F, IL-6 and TNF-α (McGeachy, M. J. et al., “The link between IL-23 and Th17 cell-mediated immune pathologies”, Semin. Immunol., 19:372-376 (2007)). These cytokines are critical in mediating the pathobiology of a number of autoimmune diseases, including rheumatoid arthritis, multiple sclerosis, inflammatory bowel disease, and lupus. IL-12, in addition to the p40 subunit in common with IL-23, contains a p35 subunit and acts through a heterodimeric receptor composed of IL-12Rβ1 and IL-12Rβ2. IL-12 is essential for Th1 cell development and secretion of IFNγ, a cytokine which plays a critical role in immunity by stimulating MHC expression, class switching of B cells to IgG subclasses, and the activation of macrophages (Gracie, J. A. et al., “Interleukin-12 induces interferon-gamma-dependent switching of IgG alloantibody subclass”, Eur. J. Immunol., 26:1217-1221 (1996); Schroder, K. et al., “Interferon-gamma: an overview of signals, mechanisms and functions”, J. Leukoc. Biol., 75(2):163-189 (2004)).

The importance of the p40-containing cytokines in autoimmunity is demonstrated by the discovery that mice deficient in either p40, p19, or IL-23R are protected from disease in models of multiple sclerosis, rheumatoid arthritis, inflammatory bowel disease, lupus and psoriasis, among others (Kyttaris, V. C. et al., “Cutting edge: IL-23 receptor deficiency prevents the development of lupus nephritis in C57BL/6-1pr/1pr mice”, J. Immunol., 184:4605-4609 (2010); Hong, K. et al., “IL-12, independently of IFN-gamma, plays a crucial role in the pathogenesis of a murine psoriasis like skin disorder”, J. Immunol., 162:7480-7491 (1999); Hue, S. et al., “Interleukin-23 drives innate and T cell-mediated intestinal inflammation”, J. Exp. Med., 203:2473-2483 (2006); Cua, D. J. et al., “Interleukin-23 rather than interleukin-12 is the critical cytokine for autoimmune inflammation of the brain”, Nature, 421:744-748 (2003); Murphy, C. A. et al., “Divergent pro- and anti-inflammatory roles for IL-23 and IL-12 in joint autoimmune inflammation”, J. Exp. Med., 198:1951-1957 (2003)).

In human disease, high expression of p40 and p19 has been measured in psoriatic lesions, and Th17 cells have been identified in active lesions in the brain from MS patients and in the gut mucosa of patients with active Crohn's disease (Lee, E. et al., “Increased expression of interleukin 23 p19 and p40 in lesional skin of patients with psoriasis vulgaris”, J. Exp. Med., 199:125-130 (2004); Tzartos, J. S. et al., “Interleukin-17 production in central nervous system infiltrating T cells and glial cells is associated with active disease in multiple sclerosis”, Am. J. Pathol., 172:146-155 (2008)). The mRNA levels of p19, p40, and p35 in active SLE patients were also shown to be significantly higher compared with those in inactive SLE patients (Huang, X. et al., “Dysregulated expression of interleukin-23 and interleukin-12 subunits in systemic lupus erythematosus patients”, Mod. Rheumatol., 17:220-223 (2007)), and T cells from lupus patients have a predominant Th1 phenotype (Tucci, M. et al., “Overexpression of interleukin-12 and T helper 1 predominance in lupus nephritis”, Clin. Exp. Immunol., 154:247-254 (2008)).

Moreover, genome-wide association studies have identified a number of loci associated with chronic inflammatory and autoimmune diseases that encode factors that function in the IL-23 and IL-12 pathways. These genes include IL23A, IL12A, IL12B, IL12RB1, IL12RB2, IL23R, JAK2, TYK2, STAT3, and STAT4 (Lees, C. W. et al., “New IBD genetics: common pathways with other diseases”, Gut, 60:1739-1753 (2011); Tao, J. H. et al., “Meta-analysis of TYK2 gene polymorphisms association with susceptibility to autoimmune and inflammatory diseases”, Mol. Biol. Rep., 38:4663-4672 (2011); Cho, J. H. et al., “Recent insights into the genetics of inflammatory bowel disease”, Gastroenterology, 140:1704-1712 (2011)).

Indeed, anti-p40 treatment, which inhibits both IL-12 and IL-23, as well as IL-23-specific anti-p19 therapies have been shown to be efficacious in the treatment of autoimmunity in diseases including psoriasis, Crohn's Disease and psoriatic arthritis (Leonardi, C. L. et al., “PHOENIX 1 study investigators. Efficacy and safety of ustekinumab, a human interleukin-12/23 monoclonal antibody, in patients with psoriasis: 76-week results from a randomized, double-blind, placebo-controlled trial (PHOENIX 1)”, Lancet, 371:1665-1674 (2008); Sandborn, W. J. et al., “Ustekinumab Crohn's Disease Study Group. A randomized trial of Ustekinumab, a human interleukin-12/23 monoclonal antibody, in patients with moderate-to-severe Crohn's disease”, Gastroenterology, 135:1130-1141 (2008); Gottlieb, A. et al., “Ustekinumab, a human interleukin 12/23 monoclonal antibody, for psoriatic arthritis: randomized, double-blind, placebo-controlled, crossover trial”, Lancet, 373:633-640 (2009)). Therefore, agents which inhibit the action of IL-12 and IL-23 may be expected to have therapeutic benefit in human autoimmune disorders.

The Type I group of interferons (IFNs), which include the IFNα members as well as IFNβ, IFNε, IFNκ and IFNω, act through a heterodimer IFNα/β receptor (IFNAR). Type I IFNs have multiple effects in both the innate and adaptive immune systems including activation of both the cellular and humoral immune responses as well as enhancing the expression and release of autoantigens (Hall, J. C. et al., “Type I interferons: crucial participants in disease amplification in autoimmunity”, Nat. Rev. Rheumatol., 6:40-49 (2010)).

In patients with systemic lupus erythematosus (SLE), a potentially fatal autoimmune disease, increased serum levels of interferon (IFN)α (a type I interferon) or increased expression of type I IFN-regulated genes (a so-called IFNα signature) in peripheral blood mononuclear cells and in affected organs has been demonstrated in a majority of patients (Bennett, L. et al., “Interferon and granulopoiesis signatures in systemic lupus erythematosus blood”, J. Exp. Med., 197:711-723 (2003); Peterson, K. S. et al., “Characterization of heterogeneity in the molecular pathogenesis of lupus nephritis from transcriptional profiles of laser-captured glomeruli”, J. Clin. Invest., 113:1722-1733 (2004)), and several studies have shown that serum IFNα levels correlate with both disease activity and severity (Bengtsson, A. A. et al., “Activation of type I interferon system in systemic lupus erythematosus correlates with disease activity but not with antiretroviral antibodies”, Lupus, 9:664-671 (2000)). A direct role for IFNα in the pathobiology of lupus is evidenced by the observation that the administration of IFNα to patients with malignant or viral diseases can induce a lupus-like syndrome. Moreover, the deletion of the IFNAR in lupus-prone mice provides high protection from autoimmunity, disease severity and mortality (Santiago-Raber, M. L. et al., “Type-I interferon receptor deficiency reduces lupus-like disease in NZB mice”, J. Exp. Med., 197:777-788 (2003)), and genome-wide association studies have identified loci associated with lupus that encode factors that function in the type I interferon pathway, including IRF5, IKBKE, TYK2, and STAT4 (Deng, Y. et al., “Genetic susceptibility to systemic lupus erythematosus in the genomic era”, Nat. Rev. Rheumatol., 6:683-692 (2010); Sandling, J. K. et al., “A candidate gene study of the type I interferon pathway implicates IKBKE and IL8 as risk loci for SLE”, Eur. J. Hum. Genet., 19:479-484 (2011)). In addition to lupus, there is evidence that aberrant activation of type I interferon-mediated pathways are important in the pathobiology of other autoimmune diseases such as Sjögren's syndrome and scleroderma (Båve, U. et al., “Activation of the type I interferon system in primary Sjögren's syndrome: a possible etiopathogenic mechanism”, Arthritis Rheum., 52:1185-1195 (2005); Kim, D. et al., “Induction of interferon-alpha by scleroderma sera containing autoantibodies to topoisomerase I: association of higher interferon-alpha activity with lung fibrosis”, Arthritis Rheum., 58:2163-2173 (2008)). Therefore, agents which inhibit the action of type I interferon responses may be expected to have therapeutic benefit in human autoimmune disorders.

Tyrosine kinase 2 (Tyk2) is a member of the Janus kinase (JAK) family of nonreceptor tyrosine kinases and has been shown to be critical in regulating the signal transduction cascade downstream of receptors for IL-12, IL-23 and type I interferons in both mice (Ishizaki, M. et al., “Involvement of Tyrosine Kinase-2 in Both the IL-12/Th1 and IL-23/Th17 Axes In vivo”, J. Immunol., 187:181-189 (2011); Prchal-Murphy, M. et al., “TYK2 kinase activity is required for functional type I interferon responses in vivo”, PLoS One, 7:e39141 (2012)) and humans (Minegishi, Y. et al., “Human tyrosine kinase 2 deficiency reveals its requisite roles in multiple cytokine signals involved in innate and acquired immunity”, Immunity, 25:745-755 (2006)). Tyk2 mediates the receptor-induced phosphorylation of members of the STAT family of transcription factors, an essential signal that leads to the dimerization of STAT proteins and the transcription of STAT-dependent pro-inflammatory genes. Tyk2-deficient mice are resistant to experimental models of colitis, psoriasis and multiple sclerosis, demonstrating the importance of Tyk2-mediated signaling in autoimmunity and related disorders (Ishizaki, M. et al., “Involvement of Tyrosine Kinase-2 in Both the IL-12/Th1 and IL-23/Th17 Axes In vivo”, J. Immunol., 187:181-189 (2011); Oyamada, A. et al., “Tyrosine kinase 2 plays critical roles in the pathogenic CD4 T cell responses for the development of experimental autoimmune encephalomyelitis”, J. Immunol., 183:7539-7546 (2009)).

In humans, individuals expressing an inactive variant of Tyk2 are protected from multiple sclerosis and possibly other autoimmune disorders (Couturier, N. et al., “Tyrosine kinase 2 variant influences T lymphocyte polarization and multiple sclerosis susceptibility”, Brain, 134:693-703 (2011)). Genome-wide association studies have shown other variants of Tyk2 to be associated with autoimmune disorders such as Crohn's Disease, psoriasis, systemic lupus erythematosus, and rheumatoid arthritis, further demonstrating the importance of Tyk2 in autoimmunity (Ellinghaus, D. et al., “Combined Analysis of Genome-wide Association Studies for Crohn Disease and Psoriasis Identifies Seven Shared Susceptibility Loci”, Am. J. Hum. Genet., 90:636-647 (2012); Graham, D. et al., “Association of polymorphisms across the tyrosine kinase gene, TYK2 in UK SLE families”, Rheumatology (Oxford), 46:927-930 (2007); Eyre, S. et al., “High-density genetic mapping identifies new susceptibility loci for rheumatoid arthritis”, Nat. Genet., 44:1336-1340 (2012)).

TYK2 inhibition may also be utilized in both solid tumors and hematologic malignancies both as a monotherapy and in combination with existing standards of care including immunotherapy.

Ex vivo studies in T-cell acute lymphoblastic leukemia (T-ALL) have shown that TYK2 is required for the survival of T-ALL, suggesting a potential direct cancer killing mechanism for TYK2 inhibitors in this indication, Sanda, T. et al. TYK2-STAT1-BCL2 Pathway Dependence in T-cell Acute Lymphoblastic Leukemia. Cancer Discov. 3, 564-577 (2013). Multiple TYK2 activating mutations in T-ALL cell lines have been detected and characterized. NPM1-TYK2 gene fusions have also been identified in a subset of cutaneous T-cell lymphomas (CTCL), and TYK2 was shown to be an oncogenic driver of transformation, Kuravi, S. et al. Functional characterization of NPM1-TYK2 fusion oncogene. Npj Precis. Oncol. 6, 3 (2022). Loss of TYK2 signaling could inhibit this transformational potential.

Effective TYK2 inhibitors have been described; however, these compounds tend to be highly polar compounds subject to high efflux ratios in standard efflux models, Wrobleski, S. T. et al. Highly selective inhibition of Tyrosine Kinase 2 (TYK2) for the treatment of autoimmune diseases: Discovery of the allosteric inhibitor BMS-986165. J. Med. Chem. 62, 8973-8995 (2019). It is well established that one pathway for multidrug resistance is increased expression of efflux transporters, Gottesman, M. M. et al. Multidrug Resistance in Cancer: Role of ATP-Dependent Transporters. Nature Rev. Cancer 2, 48-58 (2002), Fletcher, J. I. et al. ABC transporters in cancer: more than just drug efflux pumps. Nature Rev. Cancer 10, 147-156 (2010).

Therefore, compounds with lower efflux ratios in in vitro models could potentially have a greater chance of effectively treating some oncogenic indications.

In view of the conditions that may benefit by treatment involving the modulation of cytokines and/or interferons, new compounds capable of modulating cytokines and/or interferons, such as IL-12, IL-23 and/or IFNα, and methods of using these compounds may provide substantial therapeutic benefits to a wide variety of patients in need thereof.

### SUMMARY OF THE INVENTION

The invention is directed to compounds of Formula I, infra, that which are useful as modulators of IL-12, IL-23 and/or IFNα by inhibiting Tyk2-mediated signal transduction.

The present invention also provides processes and intermediates for making the compounds of the present invention.

The present invention also provides pharmaceutical compositions comprising a pharmaceutically acceptable carrier and at least one of the compounds of the present invention.

The present invention also provides a method for the modulation of IL-12, IL-23 and/or IFNα by inhibiting Tyk-2-mediated signal transduction comprising administering to a host in need of such treatment a therapeutically effective amount of at least one of the compounds of the present invention.

The present invention also provides a method for treating neurodegenerative diseases, comprising administering to a host in need of such treatment a therapeutically effective amount of at least one of the compounds of the present invention.

The present invention also provides the compounds of the present invention for use in therapy.

These and other features of the invention will be set forth in the expanded form as the disclosure continues.

In another embodiment, there is provided a pharmaceutical composition comprising one or more compounds of formula I and a pharmaceutically acceptable carrier or diluent.

The present invention is also directed to pharmaceutical compositions useful in treating diseases associated with the modulation of IL-12, IL-23 and/or IFNα by acting on Tyk-2 to cause signal transduction inhibition, comprising compounds of formula I, or pharmaceutically-acceptable salts thereof, and pharmaceutically-acceptable carriers or diluents.

The invention further relates to methods of treating diseases associated with the modulation of IL-12, IL-23, and/or IFNα, comprising administering to a patient in need of such treatment a therapeutically-effective amount of a compound according to formula I.

The present invention also provides processes and intermediates for making the compounds of the present invention.

The present invention also provides a method for treating proliferative, metabolic, allergic, autoimmune and inflammatory diseases (or use of the compounds of the present invention for the manufacture of a medicament for the treatment of these diseases), comprising administering to a host in need of such treatment a therapeutically effective amount of at least one of the compounds of the present invention.

The present invention also provides a method of treating an inflammatory or autoimmune disease (or use of the compounds of the present invention for the manufacture of a medicament for the treatment of these diseases) comprising administering to a patient in need of such treatment a therapeutically-effective amount of a compound of Formula I.

The present invention also provides a method for treating a disease (or use of the compounds of the present invention for the manufacture of a medicament for the treatment of these diseases), comprising administering to a patient in need of such treatment a therapeutically-effective amount of a compound of Formula I, wherein the disease is rheumatoid arthritis, multiple sclerosis, systemic lupus erythematosus (SLE), lupus nephritis, cutaneous lupus, inflammatory bowel disease, psoriasis, Crohn's Disease, psoriatic arthritis, Sjögren's syndrome, systemic scleroderma, ulcerative colitis, Graves' disease, discoid lupus erythematosus, adult onset Stills, systemic onset juvenile idiopathic arthritis, gout, gouty arthritis, type 1 diabetes, insulin dependent diabetes mellitus, sepsis, septic shock, Shigellosis, pancreatitis (acute or chronic), glomerulonephritis, autoimmune gastritis, diabetes, autoimmune hemolytic anemia, autoimmune neutropenia, thrombocytopenia, atopic dermatitis, myasthenia gravis, pancreatitis (acute or chronic), ankylosing spondylitis, pemphigus vulgaris, Goodpasture's disease, antiphospholipid syndrome, idiopathic thrombocytopenia, ANCA-associated vasculitis, pemphigus, Kawasaki disease, Chronic Inflammatory Demyelinating Polyneuropathy (CIDP), dermatomyositis, polymyositis, uveitis, Guillain-Barre syndrome, autoimmune pulmonary inflammation, autoimmune thyroiditis, autoimmune inflammatory eye disease, and chronic demyelinating polyneuropathy.

The present invention also provides a method of treating neurodegenerative disease (or use of the compounds of the present invention for the manufacture of a medicament for the treatment of said diseases), comprising administering to a patient in need of such treatment a therapeutically-effective amount of a compound of Formula I, wherein the disease is selected from as Alzheimer's disease, Parkinson's disease, ALS, Multiple Sclerosis (RMS and/or progressive MS, including CIS, optic neuritis, neuromyelitis optica).

The present invention also provides a method for treating a rheumatoid arthritis (or use of the compounds of the present invention for the manufacture of a medicament for the treatment of rheumatoid arthritis, comprising administering to a patient in need of such treatment a therapeutically-effective amount of a compound of Formula I.

In addition, the present invention also provides a method of treating a condition (or use of the compounds of the present invention for the manufacture of a medicament for the treatment of these conditions) comprising administering to a patient in need of such treatment a therapeutically-effective amount of a compound of Formula I, wherein the condition is selected from acute myelogenous leukemia, chronic myelogenous leukemia, metastatic melanoma, Kaposi's sarcoma, multiple myeloma, solid tumors, ocular neovasculization, and infantile haemangiomas, B cell lymphoma, systemic lupus erythematosus (SLE), rheumatoid arthritis, psoriatic arthritis, multiple vasculitides, idiopathic thrombocytopenia purpura (ITP), myasthenia gravis, allergic rhinitis, multiple sclerosis (MS), transplant rejection, Type I diabetes, membranous nephritis, inflammatory bowel disease, autoimmune hemolytic anemia, autoimmune thyroiditis, cold and warm agglutinin diseases, Evans syndrome, hemolytic uremic syndrome/thrombotic thrombocytopenia purpura (HUS/TTP), sarcoidosis, Sjögren's syndrome, peripheral neuropathies, pemphigus vulgaris and asthma.

The present invention also provides a method of treating an IL-12, IL-23, and/or IFNα mediated disease (or use of the compounds of the present invention for the manufacture of a medicament for the treatment of these diseases), comprising administering to a patient in need of such treatment a therapeutically-effective amount of a compound of formula I.

The present invention also provides a method of treating an IL-12, IL-23 and/or IFNα mediated disease (or use of the compounds of the present invention for the manufacture of a medicament for the treatment of these diseases), comprising administering to a patient in need of such treatment a therapeutically-effective amount of a compound of formula I, wherein the IL-12, IL-23 and/or IFNα mediated disease is a disease modulated by IL-12, IL-23 and/or IFNα.

The present invention also provides a method of treating diseases, comprising administering to a patient in need of such treatment a therapeutically-effective amount of a compound of formula I in combination with other therapeutic agents.

The present invention also provides the compounds of the present invention for use in therapy.

In another embodiment, compounds of formula I are selected from exemplified compounds or combinations of exemplified compounds or other embodiments herein.

In another embodiment are compounds having an IC₅₀<1000 nM in at least one of the assays described below.

The present invention may be embodied in other specific forms without departing from the spirit or essential attributes thereof. This invention encompasses all combinations of preferred aspects and/or embodiments of the invention noted herein. It is understood that any and all embodiments of the present invention may be taken in conjunction with any other embodiment or embodiments to describe additional more preferred embodiments. It is also to be understood that each individual element of the preferred embodiments is its own independent preferred embodiment. Furthermore, any element of an embodiment is meant to be combined with any and all other elements from any embodiment to describe an additional embodiment.

### DETAILED DESCRIPTION OF THE EMBODIMENTS OF THE INVENTION

In a first aspect of the present invention, there is provided a compound of formula

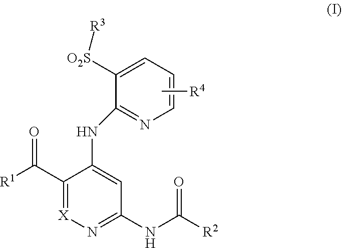

or a stereoisomer or pharmaceutically acceptable salt thereof, wherein
X is —N— or —CH—,
R¹ is C₁₋₃ alkyl or —NHCD₃;
R²is —N(CH₃)₂, —OR^{2a} or C₃₋₆ cycloalkyl substituted with 0-2 R^{2b};
R^{2a} is C₁₋₃ alkyl;
R^{2b} is F or C₁₋₃ alkyl;
R³ is C₁₋₃ fluoroalkyl or C₃₋₆ -cycloalkyl; and
R⁴ is hydrogen, halogen or C₁₋₃ alkyl.

In a second aspect of the invention, there is provided the compound of the formula

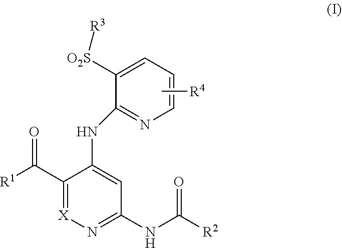

or a stereoisomer or pharmaceutically acceptable salt thereof, wherein
X is —N— or —CH—,
R¹ is C₁₋₃ alkyl or —NHCD₃;
R²is —N(CH₃)₂, —OR^{2a} or C₃₋₆ cycloalkyl substituted with 0-2 R^{2b};
R^{2a} is C₁₋₃ alkyl;
R^{2b} is F or C₁₋₃ alkyl;
R³ is CHF₂ or C₃₋₆ cycloalkyl; and
R⁴ is hydrogen, halogen or C₁₋₃ alkyl.

In a third aspect of the invention, there is provided the compound of formula II

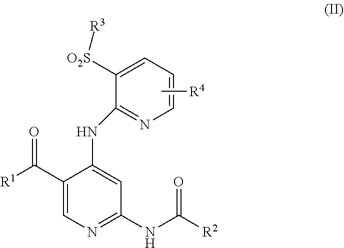

or a stereoisomer or pharmaceutically acceptable salt thereof, wherein
R¹ is C₁₋₃ alkyl or —NHCD₃;
R²is —N(CH₃)₂, —OR^{2a} or C₃₋₆ cycloalkyl substituted with 0-2 R^{2b};
R^{2a} is C₁₋₃ alkyl;
R^{2b} is F or C₁₋₃ alkyl;
R³ is CHF₂ or C₃₋₆ cycloalkyl; and
R⁴ is hydrogen, halogen or C₁₋₃ alkyl.

In a fourth aspect of the invention, there is provided the compound of formula II

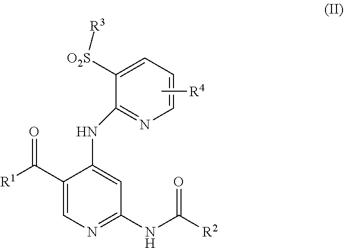

or a stereoisomer or pharmaceutically acceptable salt thereof, wherein
R¹ is C₁₋₃ alkyl;
R²is —N(CH₃)₂, —OR^{2a} or C₃₋₆ cycloalkyl substituted with 0-2 R^{2b};
R^{2a} is C₁₋₃ alkyl;
R^{2b} is F or C₁₋₃ alkyl;
R³ is CHF₂ or C₃₋₆ cycloalkyl; and
R⁴ is hydrogen, halogen or C₁₋₃ alkyl.

In a fifth aspect of the invention, there is provided the compound of formula III

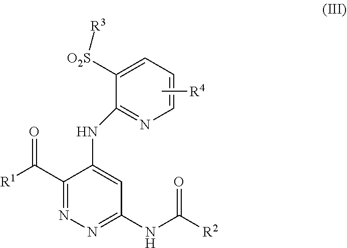

or a stereoisomer or pharmaceutically acceptable salt thereof, wherein
R¹ is C₁₋₃ alkyl or —NHCD₃;
R²is —N(CH₃)₂, —OR^{2a} or C₃₋₆ cycloalkyl substituted with 0-2 R^{2b};
R^{2a} is C₁₋₃ alkyl;
R^{2b} is F or C₁₋₃ alkyl;
R³ is CHF₂ or C₃₋₆ cycloalkyl; and
R⁴ is hydrogen, halogen or C₁₋₃ alkyl.

In a sixth aspect of the invention, there is provided the compound of formula III

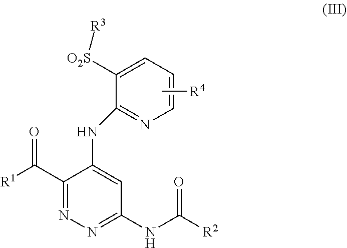

or a stereoisomer or pharmaceutically acceptable salt thereof, wherein
R¹ is —NHCD₃;
R²is —N(CH₃)₂, —OR^{2a} or C₃₋₆ cycloalkyl substituted with 0-2 R^{2b};
R^{2a} is C₁₋₃ alkyl;
R^{2b} is F or C₁₋₃ alkyl;
R³ is CHF₂ or C₃₋₆ cycloalkyl; and

R⁴ is hydrogen, halogen or C₁₋₃ alkyl.

In another aspect, there is provided a compound selected from the exemplified examples within the scope of the first aspect, or a pharmaceutically acceptable salt thereof.

In another aspect, there is provided a compound selected from any subset list of compounds within the scope of any of the above aspects.

In another aspect, there is provided a compound (IUPAC naming convention) or a pharmaceutically acceptable salt thereof, selected from
6-(cyclopropanecarboxamido)-4-((3-(cyclopropylsulfonyl)-pyridin-2-yl) amino)-N-(methyl-d₃)pyridazine-3-carboxamide,
methyl (5-((3-(cyclopropyl-sulfonyl)pyridin-2-yl)amino)-6-((methyl-d₃)-carbamoyl)-pyridazin-3-yl)-carbamate,
N-(4-((3-(cyclopropyl-sulfonyl)pyridin-2-yl)amino)-5-propionylpyridin-2-yl)cyclo-propanecarboxamide,
6-(cyclopropanecarboxamido)-4-((3-((difluoromethyl)-sulfonyl)pyridin-2-yl)amino)-N-(methyl-d3)pyridazine-3-carboxamide,
methyl (5-((3-((difluoromethyl)sulfonyl)pyridin-2-yl)amino)-6-((methyl-d₃)carbamoyl)pyridazin-3-yl)carbamate,
4-((3-((difluoromethyl)sulfonyl)pyridin-2-yl)amino)-6-(3,3-dimethylureido)-N-(methyl-d₃)pyridazine-3-carboxamide,
4-((3-((difluoromethyl)sulfonyl)pyridin-2-yl)amino)-N-(methyl-d₃)-6-(3-methyl-2-oxoimidazolidin-1-yl)pyridazine-3-carboxamide,
4-((3-((difluoromethyl)sulfonyl)pyridin-2-yl)amino)-N-(methyl-d₃)-6-((1R,2R)-2-methylcyclopropane-1-carboxamido)pyridazine-3-carboxamide,
(R)-4-((3-((difluoromethyl)sulfonyl)pyridin-2-yl)amino)-N-(methyl-d₃)-6-(spiro[2.2]pentane-1-carboxamido)pyridazine-3-carboxamide,
4-((3-((difluoromethyl)sulfonyl)pyridin-2-yl)amino)-N-(methyl-d₃)-6-((1S,2S)-2-methylcyclopropane-1-carboxamido)pyridazine-3-carboxamide,
(R)-4-((3-((difluoromethyl)sulfonyl)pyridin-2-yl)amino)-6-(2,2-dimethylcyclopropane-1-carboxamido)-N-(methyl-d₃)pyridazine-3-carboxamide,
4-((3-((difluoromethyl)sulfonyl)pyridin-2-yl)amino)-6-((1R,2S)-2-fluorocyclopropane-1-carboxamido)-N-(methyl-d₃)pyridazine-3-carboxamide,
4-((3-((difluoromethyl)sulfonyl)pyridin-2-yl)amino)-N-(methyl-d₃)-6-(2-methylcyclopropane-1-carboxamido)pyridazine-3-carboxamide,
4-((3-((difluoromethyl)sulfonyl)pyridin-2-yl)amino)-6-((6-fluoropyridin-2-yl)amino)-N-(methyl-d₃)pyridazine-3-carboxamide,
N-(4-((3-((difluoromethyl)sulfonyl)pyridin-2-yl)amino)-5-propionylpyridin-2-yl)cyclopropanecarboxamide,
3-(4-((3-((difluoromethyl)sulfonyl)pyridin-2-yl)amino)-5-propionylpyridin-2-yl)-1,1-dimethylurea,
((R)-N-(4-((3-((difluoromethyl)sulfonyl)pyridin-2-yl)amino)-5-propionylpyridin-2-yl)spiro[2.2]pentane-1-carboxamide,
(1S,2S)-N-(4-((3-((difluoromethyl)sulfonyl)pyridin-2-yl)amino)-5-propionylpyridin-2-yl)-2-methylcyclopropane-1-carboxamide,
(R)-N-(4-((3-((difluoromethyl)sulfonyl)pyridin-2-yl)amino)-5-propionylpyridin-2-yl)-2,2-dimethylcyclopropane-1-carboxamide,
(1R,2S)-N-(4-((3-((difluoromethyl)sulfonyl)pyridin-2-yl)amino)-5-propionylpyridin-2-yl)-2-fluorocyclopropane-1-carboxamide,
N-(4-((3-((difluoromethyl)sulfonyl)pyridin-2-yl)amino)-5-propionylpyridin-2-yl)-2-methylcyclopropane-1-carboxamide, and
1-(4-((3-((difluoromethyl)sulfonyl)pyridin-2-yl)amino)-6-((6-fluoropyridin-2-yl)amino)pyridin-3-yl)propan-1-one.

In another embodiment, there is provided a pharmaceutical composition comprising one or more compounds of formula I and a pharmaceutically acceptable carrier or diluent.

The present invention is also directed to pharmaceutical compositions useful in treating diseases associated with the modulation of IL-12, IL-23 and/or IFNα by acting on Tyk-2 to cause signal transduction inhibition, comprising compounds of formula I, or pharmaceutically-acceptable salts thereof, and pharmaceutically-acceptable carriers or diluents.

The invention further relates to methods of treating diseases associated with the modulation of IL-12, IL-23, and/or IFNα, comprising administering to a patient in need of such treatment a therapeutically-effective amount of a compound according to formula I.

The present invention also provides processes and intermediates for making the compounds of the present invention.

The present invention also provides a method for treating proliferative, metabolic, allergic, autoimmune and inflammatory diseases (or use of the compounds of the present invention for the manufacture of a medicament for the treatment of these diseases), comprising administering to a host in need of such treatment a therapeutically effective amount of at least one of the compounds of the present invention.

The present invention also provides a method of treating an inflammatory or autoimmune disease (or use of the compounds of the present invention for the manufacture of a medicament for the treatment of these diseases) comprising administering to a patient in need of such treatment a therapeutically-effective amount of a compound of Formula I.

The present invention also provides a method for treating a disease (or use of the compounds of the present invention for the manufacture of a medicament for the treatment of these diseases), comprising administering to a patient in need of such treatment a therapeutically-effective amount of a compound of Formula I, wherein the disease is rheumatoid arthritis, multiple sclerosis, systemic lupus erythematosus (SLE), lupus nephritis, cutaneous lupus, inflammatory bowel disease, psoriasis, Crohn's Disease, psoriatic arthritis, Sjögren's syndrome, systemic scleroderma, ulcerative colitis, Graves' disease, discoid lupus erythematosus, adult onset Stills, systemic onset juvenile idiopathic arthritis, gout, gouty arthritis, type 1 diabetes, insulin dependent diabetes mellitus, sepsis, septic shock, Shigellosis, pancreatitis (acute or chronic), glomerulonephritis, autoimmune gastritis, diabetes, autoimmune hemolytic anemia, autoimmune neutropenia, thrombocytopenia, atopic dermatitis, myasthenia gravis, pancreatitis (acute or chronic), ankylosing spondylitis, pemphigus vulgaris, Goodpasture's disease, antiphospholipid syndrome, idiopathic thrombocytopenia, ANCA-associated vasculitis, pemphigus, Kawasaki disease, Chronic Inflammatory Demyelinating Polyneuropathy (CIDP), dermatomyositis, polymyositis, uveitis, Guillain-Barre syndrome, autoimmune pulmonary inflammation, autoimmune thyroiditis, autoimmune inflammatory eye disease, and chronic demyelinating polyneuropathy.

The present invention also provides a method of treating neurodegenerative disease (or use of the compounds of the present invention for the manufacture of a medicament for the treatment of said diseases), comprising administering to a patient in need of such treatment a therapeutically-effective amount of a compound of Formula I, wherein the disease is selected from as Alzheimer's disease, Parkinson's disease, ALS, Multiple Sclerosis (RMS and/or progressive MS, including CIS, optic neuritis, neuromyelitis optica).

The present invention also provides a method for treating a rheumatoid arthritis (or use of the compounds of the present invention for the manufacture of a medicament for the treatment of rheumatoid arthritis, comprising administering to a patient in need of such treatment a therapeutically-effective amount of a compound of Formula I.

In addition, the present invention also provides a method of treating a condition (or use of the compounds of the present invention for the manufacture of a medicament for the treatment of these conditions) comprising administering to a patient in need of such treatment a therapeutically-effective amount of a compound of Formula I, wherein the condition is selected from acute myelogenous leukemia, chronic myelogenous leukemia, metastatic melanoma, Kaposi's sarcoma, multiple myeloma, solid tumors, ocular neovasculization, and infantile haemangiomas, B cell lymphoma, systemic lupus erythematosus (SLE), rheumatoid arthritis, psoriatic arthritis, multiple vasculitides, idiopathic thrombocytopenia purpura (ITP), myasthenia gravis, allergic rhinitis, multiple sclerosis (MS), transplant rejection, Type I diabetes, membranous nephritis, inflammatory bowel disease, autoimmune hemolytic anemia, autoimmune thyroiditis, cold and warm agglutinin diseases, Evans syndrome, hemolytic uremic syndrome/thrombotic thrombocytopenia purpura (HUS/TTP), sarcoidosis, Sjögren's syndrome, peripheral neuropathies, pemphigus vulgaris and asthma.

The present invention also provides a method of treating an IL-12, IL-23, and/or IFNα mediated disease (or use of the compounds of the present invention for the manufacture of a medicament for the treatment of these diseases), comprising administering to a patient in need of such treatment a therapeutically-effective amount of a compound of formula I.

The present invention also provides a method of treating an IL-12, IL-23 and/or IFNα mediated disease (or use of the compounds of the present invention for the manufacture of a medicament for the treatment of these diseases), comprising administering to a patient in need of such treatment a therapeutically-effective amount of a compound of formula I, wherein the IL-12, IL-23 and/or IFNα mediated disease is a disease modulated by IL-12, IL-23 and/or IFNα.

The present invention also provides a method of treating diseases, comprising administering to a patient in need of such treatment a therapeutically-effective amount of a compound of formula I in combination with other therapeutic agents.

The present invention also provides the compounds of the present invention for use in therapy.

In another embodiment, compounds of formula I are selected from exemplified compounds or combinations of exemplified compounds or other embodiments herein.

The present invention may be embodied in other specific forms without departing from the spirit or essential attributes thereof. This invention encompasses all combinations of preferred aspects and/or embodiments of the invention noted herein. It is understood that any and all embodiments of the present invention may be taken in conjunction with any other embodiment or embodiments to describe additional more preferred embodiments. It is also to be understood that each individual element of the preferred embodiments is its own independent preferred embodiment. Furthermore, any element of an embodiment is meant to be combined with any and all other elements from any embodiment to describe an additional embodiment.

### DETAILED DESCRIPTION OF THE INVENTION

The following are definitions of terms used in this specification and appended claims. The initial definition provided for a group or term herein applies to that group or term throughout the specification and claims, individually or as part of another group, unless otherwise indicated.

Compounds of this invention may have one or more asymmetric centers. Unless otherwise indicated, all chiral (enantiomeric and diastereomeric) and racemic forms of compounds of the present invention are included in the present invention. Many geometric isomers of olefins, C═N double bonds, and the like can also be present in the compounds, and all such stable isomers are contemplated in the present invention. Cis- and trans-geometric isomers of the compounds of the present invention are described and may be isolated as a mixture of isomers or as separated isomeric forms. The present compounds can be isolated in optically active or racemic forms. It is well known in the art how to prepare optically active forms, such as by resolution of racemic forms or by synthesis from optically active starting materials. All chiral, (enantiomeric and diastereomeric) and racemic forms and all geometric isomeric forms of a structure are intended, unless the specific stereochemistry or isomer form is specifically indicated.

When any variable (e.g., R³) occurs more than one time in any constituent or formula for a compound, its definition at each occurrence is independent of its definition at every other occurrence. Thus, for example, if a group is shown to be substituted with 0-2 R³, then said group may optionally be substituted with up to two R³ groups and R³ at each occurrence is selected independently from the definition of R³. Also, combinations of substituents and/or variables are permissible only if such combinations result in stable compounds.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents and/or variables are permissible only if such combinations result in stable compounds.

In cases wherein there are nitrogen atoms (e.g., amines) on compounds of the present invention, these can be converted to N-oxides by treatment with an oxidizing agent (e.g., MCPBA and/or hydrogen peroxides) to afford other compounds of this invention. Thus, all shown and claimed nitrogen atoms are considered to cover both the shown nitrogen and its N-oxide (N→O) derivative.

In accordance with a convention used in the art,

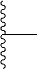

is used in structural formulas herein to depict the bond that is the point of attachment of the moiety or substituent to the core or backbone structure.

A dash “—” that is not between two letters or symbols is used to indicate a point of attachment for a substituent. For example, —CONH₂ is attached through the carbon atom.

The term “optionally substituted” in reference to a particular moiety of the compound of Formula I (e.g., an optionally substituted heteroaryl group) refers to a moiety having 0, 1, 2, or more substituents. For example, “optionally substituted alkyl” encompasses both “alkyl” and “substituted alkyl” as defined below. It will be understood by those skilled in the art, with respect to any group containing one or more substituents, that such groups are not intended to introduce any substitution or substitution patterns that are sterically impractical, synthetically non-feasible and/or inherently unstable.

As used herein, the term “at least one chemical entity” is interchangeable with the term “a compound”.

As used herein, the term “alkyl” or “alkylene” is intended to include both branched and straight-chain saturated aliphatic hydrocarbon groups having the specified number of carbon atoms. For example, “C₁₋₁₀ alkyl” (or alkylene), is intended to include C₁, C₂, C₃, C₄, C₅, C₆, C₇, C₈, C₉, and C₁₀ alkyl groups. Additionally, for example, “C₁-C₆ alkyl” denotes alkyl having 1 to 6 carbon atoms. Alkyl groups can be unsubstituted or substituted so that one or more of its hydrogens are replaced by another chemical group. Example alkyl groups include, but are not limited to, methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like.

One skilled in the field will understand that, when the designation “CO₂” is used herein, this is intended to refer to the group

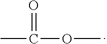

When the term “alkyl” is used together with another group, such as in “arylalkyl”, this conjunction defines with more specificity at least one of the substituents that the substituted alkyl will contain. For example, “arylalkyl” refers to a substituted alkyl group as defined above where at least one of the substituents is an aryl, such as benzyl. Thus, the term aryl(C₀₋₄)alkyl includes a substituted lower alkyl having at least one aryl substituent and also includes an aryl directly bonded to another group, i.e., aryl(C₀)alkyl.

The term “heteroarylalkyl” refers to a substituted alkyl group as defined above where at least one of the substituents is a heteroaryl.

The term “alkoxy” refers to an oxygen atom substituted by alkyl or substituted alkyl, as defined herein. For example, the term “alkoxy” includes the group —O—C₁₋₆alkyl such as methoxy, ethoxy, propoxy, isopropoxy, n-butoxy, sec-butoxy, tert-butoxy, pentoxy, 2-pentyloxy, isopentoxy, neopentoxy, hexoxy, 2-hexoxy, 3-hexoxy, 3-methylpentoxy, and the like. “Lower alkoxy” refers to alkoxy groups having one to four carbons.

It should be understood that the selections for all groups, including for example, alkoxy, thioalkyl, and aminoalkyl, will be made by one skilled in the field to provide stable compounds.

The term “substituted”, as used herein, means that any one or more hydrogens on the designated atom or group is replaced with a selection from the indicated group, provided that the designated atom's normal valence is not exceeded. When a substituent is oxo, or keto, (i.e., ═O) then 2 hydrogens on the atom are replaced. Keto substituents are not present on aromatic moieties. Unless otherwise specified, substituents are named into the core structure. For example, it is to be understood that when (cycloalkyl)alkyl is listed as a possible substituent, the point of attachment of this substituent to the core structure is in the alkyl portion. Ring double bonds, as used herein, are double bonds that are formed between two adjacent ring atoms (e.g., C═C, C═N, or N═N).

Combinations of substituents and/or variables are permissible only if such combinations result in stable compounds or useful synthetic intermediates. A stable compound or stable structure is meant to imply a compound that is sufficiently robust to survive isolation from a reaction mixture to a useful degree of purity, and subsequent formulation into an efficacious therapeutic agent. It is preferred that the presently recited compounds do not contain a N-halo, S(O)₂H, or S(O)H group.

The term “cycloalkyl” refers to cyclized alkyl groups, including mono-, bi- or poly-cyclic ring systems. C₃₋₇ cycloalkyl is intended to include C₃, C₄, C₅, C₆, and C₇ cycloalkyl groups. Example cycloalkyl groups include, but are not limited to, cyclopropyl, cyclobutyl, cyclopentyl, cyclohexyl, norbornyl, and the like. As used herein, “carbocycle” or “carbocyclic residue” is intended to mean any stable 3-, 4-, 5-, 6-, or 7-membered monocyclic or bicyclic or 7-, 8-, 9-, 10-, 11-, 12-, or 13-membered bicyclic or tricyclic ring, any of which may be saturated, partially unsaturated, unsaturated or aromatic. Examples of such carbocycles include, but are not limited to, cyclopropyl, cyclobutyl, cyclobutenyl, cyclopentyl, cyclopentenyl, cyclohexyl, cycloheptenyl, cycloheptyl, cycloheptenyl, adamantyl, cyclooctyl, cyclooctenyl, cyclooctadienyl, [3.3.0]bicyclooctane, [4.3.0]bicyclononane, [4.4.0]bicyclodecane, [2.2.2]bicyclooctane, fluorenyl, phenyl, naphthyl, indanyl, adamantyl, anthracenyl, and tetrahydronaphthyl (tetralin). As shown above, bridged rings are also included in the definition of carbocycle (e.g., [2.2.2]bicyclooctane). Preferred carbocycles, unless otherwise specified, are cyclopropyl, cyclobutyl, cyclopentyl, cyclohexyl, and phenyl. When the term “carbocycle” is used, it is intended to include “aryl”. A bridged ring occurs when one or more carbon atoms link two non-adjacent carbon atoms. Preferred bridges are one or two carbon atoms. It is noted that a bridge always converts a monocyclic ring into a bicyclic ring. When a ring is bridged, the substituents recited for the ring may also be present on the bridge.

The term “aryl” refers to monocyclic or bicyclic aromatic hydrocarbon groups having 6 to 12 carbon atoms in the ring portion, such as phenyl, and naphthyl groups, each of which may be substituted.

Accordingly, in compounds of formula I, the term “cycloalkyl” includes cyclopropyl, cyclobutyl, cyclopentyl, cyclohexyl, cycloheptyl, bicyclooctyl, etc., as well as the following ring system:

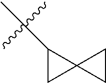 [SMILES: *C1CC12CC2]

and the like, which optionally may be substituted at any available atoms of the ring(s).

Preferred cycloalkyl groups include cyclopropyl, cyclobutyl, cyclopentyl, cyclohexyl and

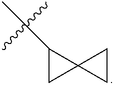 [SMILES: *C1CC12CC2]

The term “halo” or “halogen” refers to chloro, bromo, fluoro and iodo.

The term “haloalkyl” means a substituted alkyl having one or more halo substituents. For example, “haloalkyl” includes mono, bi, and trifluoromethyl.

The term “haloalkoxy” means an alkoxy group having one or more halo substituents. For example, “haloalkoxy” includes OCF3.

The terms “heterocycle”, “heterocycloalkyl”, “heterocyclo”, “heterocyclic”, or “heterocyclyl” may be used interchangeably and refer to substituted and unsubstituted 3- to 7-membered monocyclic groups, 7- to 11-membered bicyclic groups, and 10- to 15-membered tricyclic groups, in which at least one of the rings has at least one heteroatom (O, S or N), said heteroatom containing ring preferably having 1, 2, or 3 heteroatoms selected from O, S, and N. Each ring of such a group containing a heteroatom can contain one or two oxygen or sulfur atoms and/or from one to four nitrogen atoms provided that the total number of heteroatoms in each ring is four or less, and further provided that the ring contains at least one carbon atom. The nitrogen and sulfur atoms may optionally be oxidized and the nitrogen atoms may optionally be quaternized. The fused rings completing the bicyclic and tricyclic groups may contain only carbon atoms and may be saturated, partially saturated, or fully unsaturated. The heterocyclo group may be attached at any available nitrogen or carbon atom. As used herein the terms “heterocycle”, “heterocycloalkyl”, “heterocyclo”, “heterocyclic”, and “heterocyclyl” include “heteroaryl” groups, as defined below.

In addition to the heteroaryl groups described below, exemplary monocyclic heterocyclyl groups include azetidinyl, pyrrolidinyl, oxetanyl, imidazolinyl, oxazolidinyl, isoxazolinyl, thiazolidinyl, isothiazolidinyl, tetrahydrofuranyl, piperidyl, piperazinyl, 2-oxopiperazinyl, 2-oxopiperidyl, 2-oxopyrrolodinyl, 2-oxoazepinyl, azepinyl, 1-pyridonyl, 4-piperidonyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl and the like. Exemplary bicyclic heterocyclo groups include quinuclidinyl. Additional monocyclic heterocyclyl groups include

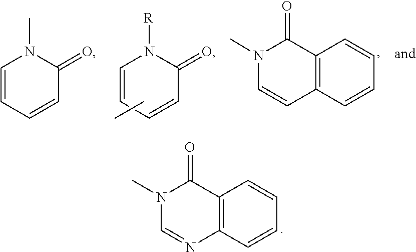

The term “heteroaryl” refers to substituted and unsubstituted aromatic 5- or 6-membered monocyclic groups, 9- or 10-membered bicyclic groups, and 11- to 14-membered tricyclic groups which have at least one heteroatom (O, S or N) in at least one of the rings, said heteroatom-containing ring preferably having 1, 2, or 3 heteroatoms selected from O, S, and N. Each ring of the heteroaryl group containing a heteroatom can contain one or two oxygen or sulfur atoms and/or from one to four nitrogen atoms provided that the total number of heteroatoms in each ring is four or less and each ring has at least one carbon atom. The fused rings completing the bicyclic and tricyclic groups may contain only carbon atoms and may be saturated, partially saturated, or unsaturated. The nitrogen and sulfur atoms may optionally be oxidized and the nitrogen atoms may optionally be quaternized. Heteroaryl groups which are bicyclic or tricyclic must include at least one fully aromatic ring but the other fused ring or rings may be aromatic or non-aromatic. The heteroaryl group may be attached at any available nitrogen or carbon atom of any ring. As valence allows, if said further ring is cycloalkyl or heterocyclo it is additionally optionally substituted with ═O (oxo).

Exemplary monocyclic heteroaryl groups include pyrrolyl, pyrazolyl, pyrazolinyl, imidazolyl, oxazolyl, isoxazolyl, thiazolyl, thiadiazolyl, isothiazolyl, furanyl, thienyl, oxadiazolyl, pyridyl, pyrazinyl, pyrimidinyl, pyridazinyl, triazinyl and the like.

Unless otherwise indicated, when reference is made to a specifically-named aryl (e.g., phenyl), cycloalkyl (e.g., cyclohexyl), heterocyclo (e.g., pyrrolidinyl, piperidinyl, and morpholinyl) or heteroaryl (e.g., tetrazolyl, imidazolyl, pyrazolyl, triazolyl, thiazolyl, and furyl) the reference is intended to include rings having 0 to 3, preferably 0 to 2, substituents selected from those recited above for the aryl, cycloalkyl, heterocyclo and/or heteroaryl groups, as appropriate.

The term “carbocyclyl” or “carbocyclic” refers to a saturated or unsaturated monocyclic or bicyclic ring in which all atoms of all rings are carbon. Thus, the term includes cycloalkyl and aryl rings. Monocyclic carbocycles have 3 to 6 ring atoms, still more typically 5 or 6 ring atoms. Bicyclic carbocycles have 7 to 12 ring atoms, e.g., arranged as a bicyclo [4,5], [5,5], [5,6] or [6,6] system, or 9 or 10 ring atoms arranged as a bicyclo [5,6] or [6,6] system. Examples of mono-and bicyclic carbocycles include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, phenyl and naphthyl. The carbocyclic ring may be substituted in which case the substituents are selected from those recited above for cycloalkyl and aryl groups.

The term “heteroatoms” shall include oxygen, sulfur and nitrogen.

When the term “unsaturated” is used herein to refer to a ring or group, the ring or group may be fully unsaturated or partially unsaturated.

Throughout the specification, groups and substituents thereof may be chosen by one skilled in the field to provide stable moieties and compounds and compounds useful as pharmaceutically acceptable compounds and/or intermediate compounds useful in making pharmaceutically-acceptable compounds.

The compounds of formula I may exist in a free form (with no ionization) or can form salts which are also within the scope of this invention. Unless otherwise indicated, reference to an inventive compound is understood to include reference to the free form and to salts thereof. The term “salt(s)” denotes acidic and/or basic salts formed with inorganic and/or organic acids and bases. In addition, the term “salt(s)” may include zwitterions (inner salts), e.g., when a compound of formula I, contains both a basic moiety, such as an amine or a pyridine or imidazole ring, and an acidic moiety, such as a carboxylic acid. Pharmaceutically acceptable (i.e., non-toxic, physiologically acceptable) salts are preferred, such as, for example, acceptable metal and amine salts in which the cation does not contribute significantly to the toxicity or biological activity of the salt. However, other salts may be useful, e.g., in isolation or purification steps which may be employed during preparation, and thus, are contemplated within the scope of the invention. Salts of the compounds of the formula I may be formed, for example, by reacting a compound of the formula I with an amount of acid or base, such as an equivalent amount, in a medium such as one in which the salt precipitates or in an aqueous medium followed by lyophilization.

Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, 2-hydroxyethanesulfonates, lactates, maleates (formed with maleic acid), methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

Exemplary basic salts include ammonium salts, alkali metal salts such as sodium, lithium, and potassium salts; alkaline earth metal salts such as calcium and magnesium salts; barium, zinc, and aluminum salts; salts with organic bases (for example, organic amines) such as trialkylamines such as triethylamine, procaine, dibenzylamine, N-benzyl-β-phenethylamine, 1-ephenamine, N,N′-dibenzylethylene-diamine, dehydroabietylamine, N-ethylpiperidine, benzylamine, dicyclohexylamine or similar pharmaceutically acceptable amines and salts with amino acids such as arginine, lysine and the like. Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others. Preferred salts include monohydrochloride, hydrogensulfate, methanesulfonate, phosphate or nitrate salts.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically-acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 18th Edition, Mack Publishing Company, Easton, PA (1990), the disclosure of which is hereby incorporated by reference.

All stereoisomers of the compounds of the instant invention are contemplated, either in admixture or in pure or substantially pure form. Stereoisomers may include compounds which are optical isomers through possession of one or more chiral atoms, as well as compounds which are optical isomers by virtue of limited rotation about one or more bonds (atropisomers). The definition of compounds according to the invention embraces all the possible stereoisomers and their mixtures. It very particularly embraces the racemic forms and the isolated optical isomers having the specified activity. The racemic forms can be resolved by physical methods, such as, for example, fractional crystallization, separation or crystallization of diastereomeric derivatives or separation by chiral column chromatography. The individual optical isomers can be obtained from the racemates from the conventional methods, such as, for example, salt formation with an optically active acid followed by crystallization.

The present invention is intended to include all isotopes of atoms occurring in the present compounds. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent otherwise employed.

Prodrugs and solvates of the inventive compounds are also contemplated. The term “prodrug” denotes a compound which, upon administration to a subject, undergoes chemical conversion by metabolic or chemical processes to yield a compound of the formula I, and/or a salt and/or solvate thereof. Any compound that will be converted in vivo to provide the bioactive agent (i.e., the compound for formula I) is a prodrug within the scope and spirit of the invention. For example, compounds containing a carboxy group can form physiologically hydrolyzable esters which serve as prodrugs by being hydrolyzed in the body to yield formula I compounds per se. Such prodrugs are preferably administered orally since hydrolysis in many instances occurs principally under the influence of the digestive enzymes. Parenteral administration may be used where the ester per se is active, or in those instances where hydrolysis occurs in the blood. Examples of physiologically hydrolyzable esters of compounds of formula I include C₁₋₆alkylbenzyl, 4-methoxybenzyl, indanyl, phthalyl, methoxymethyl, C₁₋₆alkanoyloxy-C₁₋₆alkyl, e.g., acetoxymethyl, pivaloyloxymethyl or propionyloxymethyl, C₁₋₆alkoxycarbonyloxy-C₁₋₆alkyl, e.g., methoxycarbonyl-oxymethyl or ethoxycarbonyloxymethyl, glycyloxymethyl, phenylglycyloxymethyl, (5-methyl-2-oxo-1,3-dioxolen-4-yl)-methyl and other well known physiologically hydrolyzable esters used, for example, in the penicillin and cephalosporin arts. Such esters may be prepared by conventional techniques known in the art.

Various forms of prodrugs are well known in the art and are described in Rautio, J. et al., Nature Review Drug Discovery, 17, 559-587 (2018).

Compounds of the formula I and salts thereof may exist in their tautomeric form, in which hydrogen atoms are transposed to other parts of the molecules and the chemical bonds between the atoms of the molecules are consequently rearranged. It should be understood that the all tautomeric forms, insofar as they may exist, are included within the invention. Additionally, inventive compounds may have trans- and cis-isomers.

It should further be understood that solvates (e.g., hydrates) of the compounds of Formula I are also with the scope of the present invention. Methods of solvation are generally known in the art.

### UTILITY

The compounds of the invention modulate IL-23-stimulated and IFNα-stimulated cellular functions, including gene transcription. Other types of cellular functions that may be modulated by the compounds of the instant invention include, but are not limited to, IL-12-stimulated responses.

Accordingly, compounds of formula I have utility in treating conditions associated with the modulation of the function of IL-23 and/or IFNα, and particularly the selective inhibition of function of IL-23, IL-12 and/or IFNα, by acting on Tyk2 to mediate signal transduction. Such conditions include IL-23-, IL-12-or IFNα-associated diseases in which pathogenic mechanisms are mediated by these cytokines and the subsequent activation of the Tyk2 pathway with subsequent pro-inflammatory responses which may occur in the peripheral and/or central compartments.

As used herein, the terms “treating” or “treatment” encompass the treatment of a disease state in a mammal, particularly in a human, and include: (a) preventing or delaying the occurrence of the disease state in a mammal, in particular, when such mammal is predisposed to the disease state but has not yet been diagnosed as having it; (b) inhibiting the disease state, i.e., arresting or slowing its development; and/or (c) achieving a full or partial reduction of the symptoms or disease state, and/or alleviating, ameliorating, lessening, or curing the disease or disorder and/or its symptoms.

In view of their activity as modulators of IL-23-, IL-12 and/or IFNα-stimulated cellular responses, compounds of Formula I are useful in treating IL-23-, IL-12-and/or IFNα-associated diseases including, but not limited to, inflammatory diseases such as Crohn's disease, ulcerative colitis, asthma, graft versus host disease, allograft rejection, chronic obstructive pulmonary disease; autoimmune diseases such as Graves' disease, rheumatoid arthritis, systemic lupus erythematosus, cutaneous lupus, lupus nephritis, discoid lupus erythematosus, psoriasis; auto-inflammatory diseases including CAPS, TRAPS, FMF, adult onset stills, systemic onset juvenile idiopathic arthritis, gout, gouty arthritis; metabolic diseases including type 2 diabetes, atherosclerosis, myocardial infarction; destructive bone disorders such as bone resorption disease, osteoarthritis, osteoporosis, multiple myeloma-related bone disorder; proliferative disorders such as acute myelogenous leukemia, chronic myelogenous leukemia; angiogenic disorders such as angiogenic disorders including solid tumors, ocular neovasculization, and infantile haemangiomas; infectious diseases such as sepsis, septic shock, and Shigellosis; neurodegenerative diseases such as Alzheimer's disease, Parkinson's disease, ALS, Multiple Sclerosis (RMS and/or progressive MS, including CIS, optic neuritis, neuromyelitis optica), cerebral ischemias or neurodegenerative disease caused by traumatic injury, oncologic and viral diseases such as metastatic melanoma, Kaposi's sarcoma, multiple myeloma, and HIV infection and CMV retinitis, AIDS, respectively.

More particularly, the specific conditions or diseases that may be treated with the inventive compounds include, without limitation, pancreatitis (acute or chronic), asthma, allergies, adult respiratory distress syndrome, chronic obstructive pulmonary disease, glomerulonephritis, rheumatoid arthritis, systemic lupus erythematosis, cutaneous lupus, lupus nephritis, discoid lupus erythematosus, scleroderma, chronic thyroiditis, Graves' disease, autoimmune gastritis, diabetes, autoimmune hemolytic anemia, autoimmune neutropenia, thrombocytopenia, atopic dermatitis, chronic active hepatitis, myasthenia gravis, multiple sclerosis, inflammatory bowel disease, ulcerative colitis, Crohn's disease, psoriasis, graft vs. host disease, inflammatory reaction induced by endotoxin, tuberculosis, atherosclerosis, muscle degeneration, cachexia, psoriatic arthritis, Reiter's syndrome, gout, traumatic arthritis, rubella arthritis, acute synovitis, pancreatic β-cell disease; diseases characterized by massive neutrophil infiltration; rheumatoid spondylitis, gouty arthritis and other arthritic conditions, cerebral malaria, chronic pulmonary inflammatory disease, silicosis, pulmonary sarcoidosis, bone resorption disease, allograft rejections, fever and myalgias due to infection, cachexia secondary to infection, keloid formation, scar tissue formation, ulcerative colitis, pyresis, influenza, osteoporosis, osteoarthritis, acute myelogenous leukemia, chronic myelogenous leukemia, metastatic melanoma, Kaposi's sarcoma, multiple myeloma, sepsis, septic shock, and Shigellosis; Alzheimer's disease, Parkinson's disease, Multiple Sclerosis (RMS and/or progressive MS, including CIS, optic neuritis, neuromyelitis optica), cerebral ischemias or neurodegenerative disease caused by traumatic injury; angiogenic disorders including solid tumors, ocular neovascularization, and infantile haemangiomas; viral diseases including acute hepatitis infection (including hepatitis A, hepatitis B and hepatitis C), HIV infection and CMV retinitis, AIDS, ARC or malignancy, and herpes; stroke, myocardial ischemia, ischemia in stroke heart attacks, organ hypoxia, vascular hyperplasia, cardiac and renal reperfusion injury, thrombosis, cardiac hypertrophy, thrombin-induced platelet aggregation, endotoxemia and/or toxic shock syndrome, conditions associated with prostaglandin endoperoxidase syndase-2, and pemphigus vulgaris. Preferred methods of treatment are those wherein the condition is selected from Alzheimer's disease, Parkinson's disease, ALS, Multiple Sclerosis (RMS and/or progressive MS, including CIS, optic neuritis, neuromyelitis optica),

When the terms “IL-23-, IL-12- and/or IFNα-associated condition” or “IL-23-, IL-12- and/or IFNα-associated disease or disorder” are used herein, each is intended to encompass all of the conditions identified above as if repeated at length, as well as any other condition that is affected by IL-23, IL-12 and/or IFNα.

The present invention thus provides methods for treating such conditions, comprising administering to a subject in need thereof a therapeutically effective amount of at least one compound of Formula I or a salt thereof. “Therapeutically effective amount” is intended to include an amount of a compound of the present invention that is effective when administered alone or in combination to inhibit IL-23, IL-12 and/or IFNα function and/or treat diseases.

The methods of treating IL-23-, IL-12 and/or IFNα-associated conditions may comprise administering compounds of Formula I alone or in combination with each other and/or other suitable therapeutic agents useful in treating such conditions. Accordingly, “therapeutically effective amount” is also intended to include an amount of the combination of compounds claimed that is effective to inhibit IL-23, IL-12 and/or IFNα function and/or treat diseases associated with IL-23, IL-12 and/or IFNα.

Exemplary of such other therapeutic agents include corticosteroids, rolipram, calphostin, cytokine-suppressive anti-inflammatory drugs (CSAIDs), Interleukin-10, glucocorticoids, salicylates, nitric oxide, and other immunosuppressants; nuclear translocation inhibitors, such as deoxyspergualin (DSG); non-steroidal anti-inflammatory drugs (NSAIDs) such as ibuprofen, celecoxib and rofecoxib; steroids such as prednisone or dexamethasone; antiviral agents such as abacavir; antiproliferative agents such as methotrexate, leflunomide, FK506 (tacrolimus, PROGRAF®); anti-malarials such as hydroxychloroquine; cytotoxic drugs such as azathiprine and cyclophosphamide; TNF-α inhibitors such as tenidap, anti-TNF antibodies or soluble TNF receptor, and rapamycin (sirolimus or RAPAMUNE®) or derivatives thereof.

The above other therapeutic agents, when employed in combination with the compounds of the present invention, may be used, for example, in those amounts indicated in the Physicians' Desk Reference (PDR) or as otherwise determined by one of ordinary skill in the art. In the methods of the present invention, such other therapeutic agent(s) may be administered prior to, simultaneously with, or following the administration of the inventive compounds. The present invention also provides pharmaceutical compositions capable of treating IL-23-, IL-12- or IFNα-associated conditions by inhibiting Tyk2-mediated signal transduction, including IL-23-, IL-12- and/or IFNα-mediated diseases, as described above.

The inventive compositions may contain other therapeutic agents as described above and may be formulated, for example, by employing conventional solid or liquid vehicles or diluents, as well as pharmaceutical additives of a type appropriate to the mode of desired administration (e.g., excipients, binders, preservatives, stabilizers, flavors, etc.) according to techniques such as those well known in the art of pharmaceutical formulation.

Accordingly, the present invention further includes compositions comprising one or more compounds of Formula I and a pharmaceutically acceptable carrier.

A “pharmaceutically acceptable carrier” refers to media generally accepted in the art for the delivery of biologically active agents to animals, in particular, mammals. Pharmaceutically acceptable carriers are formulated according to a number of factors well within the purview of those of ordinary skill in the art. These include without limitation the type and nature of the active agent being formulated; the subject to which the agent-containing composition is to be administered; the intended route of administration of the composition; and, the therapeutic indication being targeted. Pharmaceutically acceptable carriers include both aqueous and non-aqueous liquid media, as well as a variety of solid and semi-solid dosage forms. Such carriers can include a number of different ingredients and additives in addition to the active agent, such additional ingredients being included in the formulation for a variety of reasons, e.g., stabilization of the active agent, binders, etc., well known to those of ordinary skill in the art. Descriptions of suitable pharmaceutically acceptable carriers, and factors involved in their selection, are found in a variety of readily available sources such as, for example, Remington's Pharmaceutical Sciences, 17th Edition (1985), which is incorporated herein by reference in its entirety.

The compounds of Formula I may be administered by any means suitable for the condition to be treated, which may depend on the need for site-specific treatment or quantity of drug to be delivered. Topical administration is generally preferred for skin-related diseases, and systematic treatment preferred for cancerous or pre-cancerous conditions, although other modes of delivery are contemplated. For example, the compounds may be delivered orally, such as in the form of tablets, capsules, granules, powders, or liquid formulations including syrups; topically, such as in the form of solutions, suspensions, gels or ointments; sublingually; bucally; parenterally, such as by subcutaneous, intravenous, intramuscular or intrasternal injection or infusion techniques (e.g., as sterile injectable aq. or non-aq. solutions or suspensions); nasally such as by inhalation spray; topically, such as in the form of a cream or ointment; rectally such as in the form of suppositories; or liposomally. Dosage unit formulations containing non-toxic, pharmaceutically acceptable vehicles or diluents may be administered. The compounds may be administered in a form suitable for immediate release or extended release. Immediate release or extended release may be achieved with suitable pharmaceutical compositions or, particularly in the case of extended release, with devices such as subcutaneous implants or osmotic pumps.

Exemplary compositions for topical administration include a topical carrier such as PLASTIBASE® (mineral oil gelled with polyethylene).

Exemplary compositions for oral administration include suspensions which may contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which may contain, for example, microcrystalline cellulose, dicalcium phosphate, starch, magnesium stearate and/or lactose and/or other excipients, binders, extenders, disintegrants, diluents and lubricants such as those known in the art. The inventive compounds may also be orally delivered by sublingual and/or buccal administration, e.g., with molded, compressed, or freeze-dried tablets. Exemplary compositions may include fast-dissolving diluents such as mannitol, lactose, sucrose, and/or cyclodextrins. Also included in such formulations may be high molecular weight excipients such as celluloses (AVICEL®) or polyethylene glycols (PEG); an excipient to aid mucosal adhesion such as hydroxypropyl cellulose (HPC), hydroxypropyl methyl cellulose (HPMC), sodium carboxymethyl cellulose (SCMC), and/or maleic anhydride copolymer (e.g., GANTREZ®); and agents to control release such as polyacrylic copolymer (e.g., CARBOPOL 934®). Lubricants, glidants, flavors, coloring agents and stabilizers may also be added for ease of fabrication and use.

Exemplary compositions for nasal aerosol or inhalation administration include solutions which may contain, for example, benzyl alcohol or other suitable preservatives, absorption promoters to enhance absorption and/or bioavailability, and/or other solubilizing or dispersing agents such as those known in the art.

Exemplary compositions for parenteral administration include injectable solutions or suspensions which may contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid.

Exemplary compositions for rectal administration include suppositories which may contain, for example, suitable non-irritating excipients, such as cocoa butter, synthetic glyceride esters or polyethylene glycols, which are solid at ordinary temperatures but liquefy and/or dissolve in the rectal cavity to release the drug.

The therapeutically-effective amount of a compound of the present invention may be determined by one of ordinary skill in the art, and includes exemplary dosage amounts for a mammal of from about 0.05 to 1000 mg/kg; 1-1000 mg/kg; 1-50 mg/kg; 5-250 mg/kg; 250-1000 mg/kg of body weight of active compound per day, which may be administered in a single dose or in the form of individual divided doses, such as from 1 to 4 times per day. It will be understood that the specific dose level and frequency of dosage for any particular subject may be varied and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition. Preferred subjects for treatment include animals, most preferably mammalian species such as humans, and domestic animals such as dogs, cats, horses, and the like. Thus, when the term “patient” is used herein, this term is intended to include all subjects, most preferably mammalian species that are affected by modulation of IL-23, IL-12 and/or IFNα-mediated functions.

### METHODS OF PREPARATION

The compounds of the present invention can be prepared in a number of ways well known to one skilled in the art of organic synthesis. The compounds of the present invention can be synthesized using the methods described below, together with synthetic methods known in the art of synthetic organic chemistry, or variations thereon as appreciated by those skilled in the art. Preferred methods include, but are not limited to, those described below. All references cited herein are hereby incorporated in their entirety by reference.

The compounds of this invention may be prepared using the reactions and techniques described in this section. The reactions are performed in solvents appropriate to the reagents and materials employed and are suitable for the transformations being effected. Also, in the description of the synthetic methods described below, it is to be understood that all proposed reaction conditions, including choice of solvent, reaction atmosphere, reaction temperature, duration of the experiment and work up procedures, are chosen to be the conditions standard for that reaction, which should be readily recognized by one skilled in the art. It is understood by one skilled in the art of organic synthesis that the functionality present on various portions of the molecule must be compatible with the reagents and reactions proposed. Such restrictions to the substituents that are compatible with the reaction conditions will be readily apparent to one skilled in the art and alternate methods must then be used. This will sometimes require a judgment to modify the order of the synthetic steps or to select one particular process scheme over another in order to obtain a desired compound of the invention. It will also be recognized that another major consideration in the planning of any synthetic route in this field is the judicious choice of the protecting group used for protection of the reactive functional groups present in the compounds described in this invention. An authoritative account describing the many alternatives to the trained practitioner is Greene and Wuts (Protective Groups In Organic Synthesis, Third Edition, Wiley and Sons, 1999).

The key intermediates shown in FIG. 1 can be assembled to give compound 1 in a variety of ways known to one skilled in the art of synthetic organic chemistry.

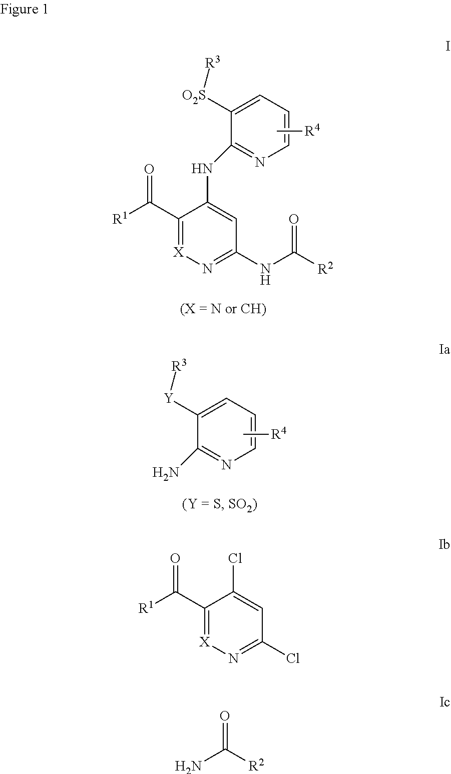

Scheme 1 shows how one skilled in the art of organic synthesis can couple intermediates of general formula Ib (refer to Moslin, et. al., J. Med. Chem 2019, 62, 8953-8972 or U.S. Pat. No. 9,505,748) with intermediates of general formula Ia to provide intermediates of general formula II. The reaction involves mixing the two reagents in an appropriate aprotic solvent, particularly THF or 2-methyl-THF at between 0° C. and 50° C. depending on the particular Ia and adding the appropriate base, particularly, lithium hexamethyldisilazide, sodium hexamethyldisilazide, potassium hexamethyldisilazide or sodium hydride.

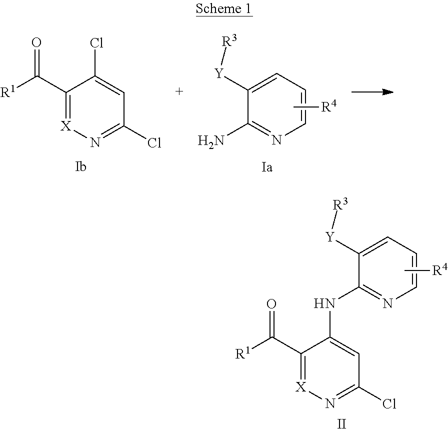

Scheme 2 shows how one skilled in the art of organic synthesis can couple compound II to the appropriate substrate Ic to produce compounds of general formula 1. The process involves the coupling of compounds of general formula II with primary amides of general formula Ic under transition metal catalyzed conditions. In particular, favorable conditions for this reaction involve employing a Buchwald type coupling, using Pd₂(dba)₃, as catalyst, 1,1′-bis(dicyclohexylphosphino)ferrocene as the ligand and Cs₂CO₃ as the base in 1,4-dioxane as solvent, at elevated temperatures. This catalyst/ligand//base system can be altered in ways known to those skilled in the art.

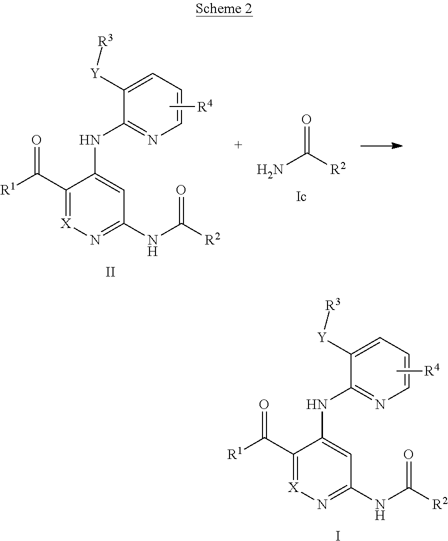

Conversion of a penultimate sulfide (I, Y=S) can be accomplished using a variety of oxidation conditions such as oxone or sodium tungstate with hydrogen peroxide. In the event that oxidation of one of the pyridine/pridazine nitrogens also occurs the desired product can be formed via a subsequent borane mediated reduction to provide the desired I (Y=SO₂)

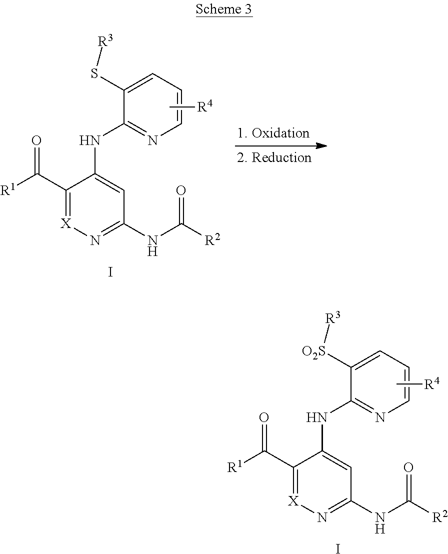

### Preparation

All reagents purchased from commercial sources were used without further purification unless otherwise noted. All reactions involving air or moisture sensitive reagents were performed under an inert atmosphere. Proton and carbon magnetic resonance (¹H and ¹³C NMR) spectra were recorded either on a Bruker Avance 400 or a JEOL Eclipse 500 spectrometer and are reported in ppm relative to the reference solvent of the sample in which they were run. HPLC and LCMS analyses were conducted using a Shimadzu LC-10AS liquid chromatograph and a SPDUV-vis detector at 220 or 254 nm with the MS detection performed with a Micromass Platform LC spectrometer. GCMS analyses were conducted using a GC (7890B)-MS(5977B) from Agilent technologies.

### Analytical Methods

### LCMS—Method A

Linear gradient of 2% to 40% solvent B over 4 minutes with 0.6-minute hold at 100% B and

followed by 0.1-minute gradient to 20% B and a 0.3-minute hold at 20% B.

Solvent A: 5 mM Ammonium formate pH 3.3: ACN (98:02)

Solvent B: ACN: Buffer (98:02)

Flow Rate: 1.0 ml/min; Column: Kinetex XB-C₁₈ (75×3.0) mm, 2.6μm

Ultraviolet (“UV”) visualization at 220 nanometers (“nm”).

### LCMS—Method B

Linear gradient of 20% to 100% solvent B over 4 minutes with 0.6-minute hold at 100% B and

followed by 0.1-minute gradient to 20% B and a 0.3-minute hold at 20% B.

Solvent A: 5 mM Ammonium formate pH 3.3: ACN (98:02)

Solvent B: ACN: Buffer (98:02)

Flow Rate: 1.0 ml/min; Column: Kinetex XB-C₁₈ (75×3.0) mm, 2.6μm

Ultraviolet (“UV”) visualization at 220 nanometers (“nm”).

### LCMS—Method C

Linear gradient of 20% to 98% solvent B over 1.5 minutes with 0.5-minute hold at 98% B and

followed by 0.1-minute gradient to 20% B and a 0.4-minute hold at 20% B

Solvent A: 0.1% TFA in H₂O

Solvent B: 0.1% TFA in ACN

Flow Rate: 0.7 ml/min; Column: Aquity Uplc BEH C₁₈ (50×2.1) mm, 1.7μm

Ultraviolet (“UV”) visualization at 220 nanometers (“nm”).

### LCMS—Method D

Linear gradient of 20% to 98% solvent B over 1.5 minutes with 0.5-minute hold at 98% B and

followed by 0.1-minute gradient to 20% B and a 0.4-minute hold at 20% B

Solvent A: 5 mM Ammonium formate pH 3.3: ACN (98:02)

Solvent B: ACN: Buffer (98:02).

Flow Rate: 0.7 ml/min; Column: Aquity Uplc BEH C₁₈ (50×3.0) mm, 1.7μm

Ultraviolet (“UV”) visualization at 220 nanometers (“nm”).

The following abbreviations may be relevant for the application.

Abbreviations

| | |
|---|---|
| ACN/MeCN | acetonitrile |
| AcOH | acetic acid |
| aq | aqueous |
| BINAP | ([1,1′-binaphthalene]-2,2′-diyl)bis(diphenylphosphane) |
| DCM | dichloromethane |
| DCPF | 1,1′-bis(dicyclohexylphosphino)ferrocene |
| DIAD | diisopropyl azodicarboxylate |
| DIPEA | diisopropyl ethyl amine |
| DMF | dimethylformamide |
| DMSO | dimethyl sulfoxide |
| EA | ethyl acetate |
| equiv | equivalents |
| ESI | electrospray ionization |
| EtOAc | ethyl acetate |
| EtOH | ethanol |
| h | hour(s) |
| Hex | hexanes |
| HPLC | high performance liquid chromatography |
| LCMS | liquid chromatography mass spectrometry |
| LiHMDS | lithium bis(trimethylsilyl)amide |
| MeOH | methanol |
| min | min(s) |
| Pd(dppf)Cl₂ | [1,1′-bis(diphenylphosphino)ferrocene]dichloropalladium(II) |
| Pd₂(dba)₃ | tris(dibenzylideneacetone)dipalladium(0) |
| PE | petroleum ether |
| Ph | phenyl |
| PPh₃ | triphenylphosphine |
| RP | reverse phase |
| RT | retention time |
| sat. | saturated |
| TFA | trifluoroacetic acid |
| THF | tetrahydrofuran |
| Ti(OiPr)₄ | titanium(IV) isopropoxide |
| TLC | thin layer chromatography |
| TMSCHN₂ | diazomethyl(trimethyl)silane |

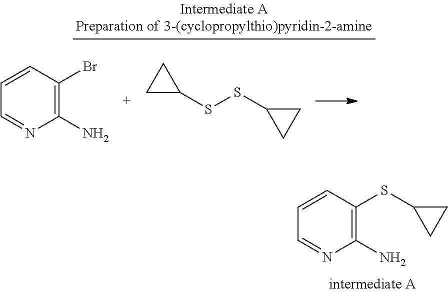 [SMILES: Nc1ncccc1Br ; C1CC1SSC1CC1 ; Nc1ncccc1SC1CC1]

To a well stirred solution of 3-bromopyridin-2-amine (0.25 g, 1.445 mmol) in THF (5 mL) in a 100 mL two neck round bottom flask was added nBuLi (3.61 ml, 5.78 mmol) dropwise at −78° C. and stirred for 15 min, then 1,2-dicyclopropyldisulfane (0.423 g, 2.89 mmol) in THF (5 mL) was added. The reaction mixture was allowed to slowly warm up to room temperature and stirred for 30 min. After 30 min, the reaction mixture was quenched with saturated aqueous ammonium chloride solution (5 mL). The reaction mixture was partitioned between EtOAc (100 mL) and water (100 mL). The organic layer was washed with brine (100 mL), dried over anhydrous sodium sulfate, and filtered. The solvent was evaporated under reduced pressure. The crude material was directly used in next step.

MS (M+1) m/z: 167 (M+H)+; LC retention time 0.572 min [Method A].

### EXAMPLE 1

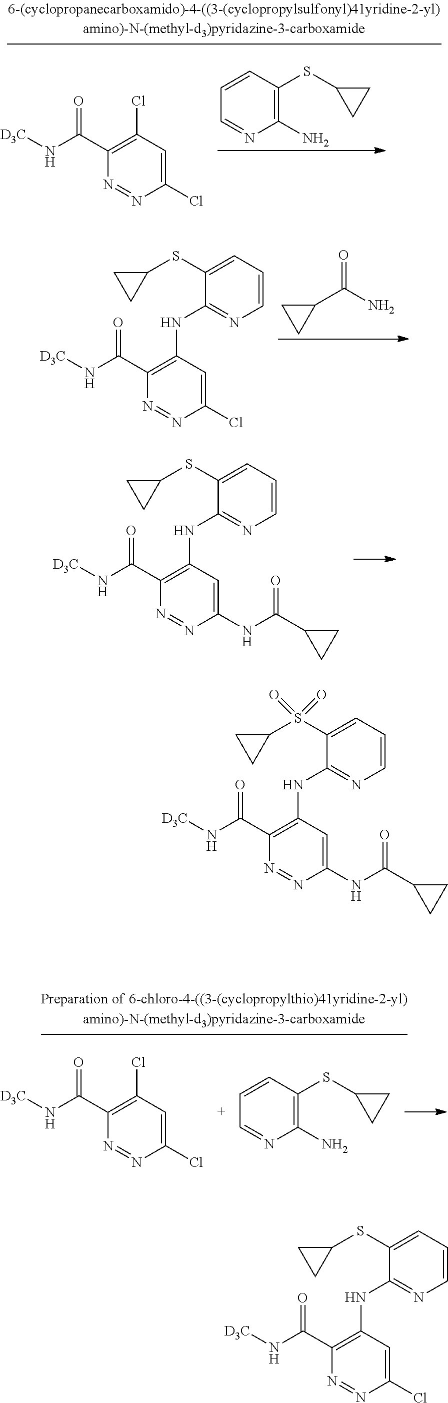

To a cooled (0° C.) solution of 3-(cyclopropylthio)pyridin-2-amine (0.05 g, 0.301 mmol) in THF (5 mL) in a 50 mL three neck round bottom flask was added 4,6-dichloro-N-(methyl-d₃)pyridazine-3-carboxamide (0.063 g, 0.301 mmol) and the mixture was stirred for 5 minutes, then LiHMDS (0.602 mL, 0.602 mmol) was added dropwise over 5 minutes. The reaction was stirred at room temperature for 30 minutes, monitored by TLC. After complete consumption of starting material, reaction mixture was quenched with saturated aqueous ammonium chloride solution (15 mL). The reaction mixture was partitioned between EtOAc (100 mL) and water (100 mL). The organic layer was washed with brine (50 mL), dried over anhydrous sodium sulfate, filtered and concentrated to afford a brown solid (100 mg). The crude solid compound was triturated with n-pentane: diethyl ether (1:1) to give 6-chloro-4-((3-(cyclopropylthio)pyridin-2-yl)amino)-N-(methyl-d₃)pyridazine-3-carboxamide (60 mg, 0.094 mmol, 31.2% yield) as an pale brown solid.

MS (M+1) m/z: 339 (M+H)+; LC retention time 2.775 min [Method A].

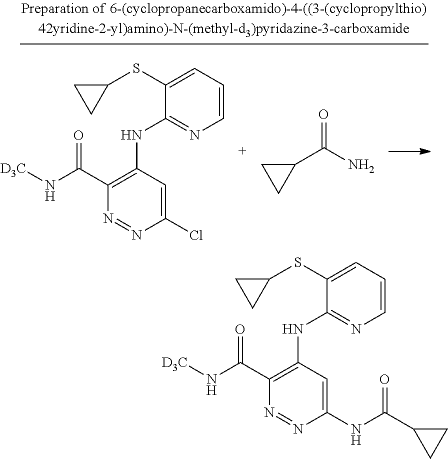 [SMILES: [2H]C([2H])([2H])NC(=O)c1nnc(Cl)cc1Nc1ncccc1SC1CC1 ; NC(=O)C1CC1 ; [2H]C([2H])([2H])NC(=O)c1nnc(NC(=O)C2CC2)cc1Nc1ncccc1SC1CC1]

To a well stirred solution of 6-chloro-4-((3-(cyclopropylthio)pyridin-2-yl)amino)-N-(methyl-d₃) pyridazine-3-carboxamide (60 mg, 0.177 mmol) in 1,4-dioxane (4 mL) in a 20 mL sealed vial were added cyclopropanecarboxamide (15.07 mg, 0.177 mmol) and Pd₂(dba)₃ (162 mg, 0.177 mmol), DCPF (102 mg, 0.177 mmol) followed by Cs₂CO₃(57.7 mg, 0.177 mmol). The reaction mixture was degassed with N₂ for 5 min. The resultant reaction mixture was heated to 110° C. for 3h, monitoring by TLC. After completion of the reaction, the mixture was diluted with ethyl acetate (100 mL), filtrated through a celite pad and the pad was thoroughly washed with ethyl acetate (100 mL). Filtrate was washed with water (2×50 mL) followed by brine (50 mL) and dried over Na₂SO₄. Solvent was evaporated under reduced pressure and the resultant crude residue was purified by reverse phase column (C18) using 45-50% water (0.1 ammonium acetate) in acetonitrile to give 6-(cyclopropanecarboxamido)-4-((3-(cyclopropylthio)pyridin-2-yl)amino)-N-(methyl-d₃) pyridazine-3-carboxamide (10 mg, 0.023 mmol, 12.83% yield) as an off-white solid.

MS (M+1) m/z: 388 (M+H)+; LC retention time 2.550 min [Method A].

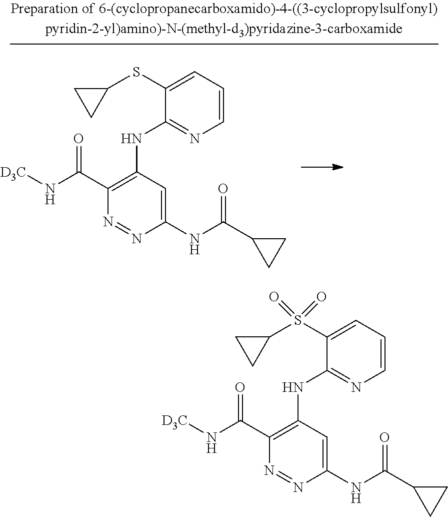

To a well stirred solution of 6-(cyclopropanecarboxamido)-4-((3-(cyclopropylthio)pyridin-2-yl)amino)-N-(methyl-d₃)pyridazine-3-carboxamide (10 mg, 0.026 mmol) in methanol (5 mL):water (0.5 mL) in a 10 mL two neck round bottom flask was added oxone (79 mg, 0.129 mmol). The resulting mixture was stirred at room temperature for 24h. After completion of the reaction, the mixture was diluted with DCM (50 mL), filtered through a celite pad and the pad was thoroughly washed with DCM (50 mL). The filtrate was washed with water (2×50 mL) followed by brine (50 mL) and dried over Na₂SO₄. The solvent was evaporated off under reduced pressure and the resultant crude residue was purified by reverse phase column (C18) purification using 45-50% water (0.1% ammonium formate) in acetonitrile to give 6-(cyclopropanecarboxamido)-4-((3-(cyclopropylsulfonyl)pyridin-2-yl)amino)-N-(methyl-d₃) pyridazine-3-carboxamide (4 mg, 9.48 μmol, 36.7% yield) as a off-white solid.

Analytical data:
HPLC-Purity: 99.455%. Method info: Column: Kinetex Biphenyl (100×4.6) mm, 2.6μm
Mobile phase A: 0.05% TFA in water:ACN (95:5) Mobile phase B: ACN:0.05% TFA in water (95:5) Flow: 1.0 mL/min, RT—6.786 min.
LC-MS-Purity: 98.960%; m/z (E+): 420.2 [M+H]+. Method info: Column: Kinetex XB- C18 (75×3) mm, 2.6μm Mobile Phase A: 5 mM Ammonium formate pH 3.3:ACN (98:02) Mobile Phase B: ACN: Buffer (98:02) Flow Rate: 1.0 ml/min, RT—1.763 min.

### ¹H-NMR (400 MHz, DMSO-d6): δ 12.10 (s, 1H), 11.45 (s, 1H), 9.42 (s, 1H), 9.20 (s, 1H), 8.61 (d, J=2.80 Hz, 1H), 8.24 (t, J=6.00 Hz, 1H), 7.34-7.31 (m, 1H), 3.10 (t, J=4.40 Hz, 1H), 2.12 (t, J=5.60 Hz, 1H), 1.18-1.06 (m, 4H), 0.87-0.86 (m, 4H).

### EXAMPLE 2

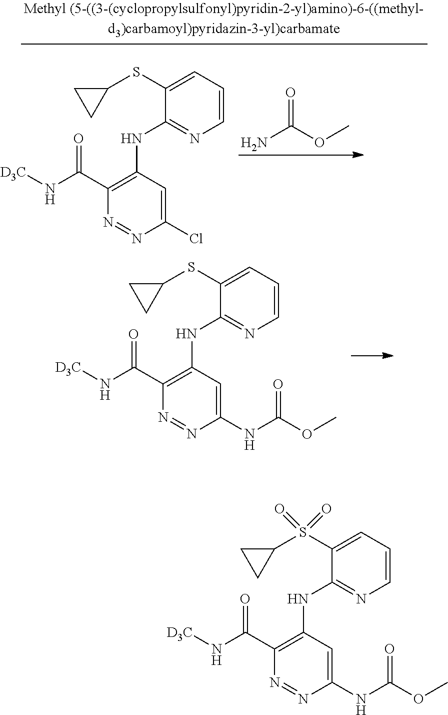

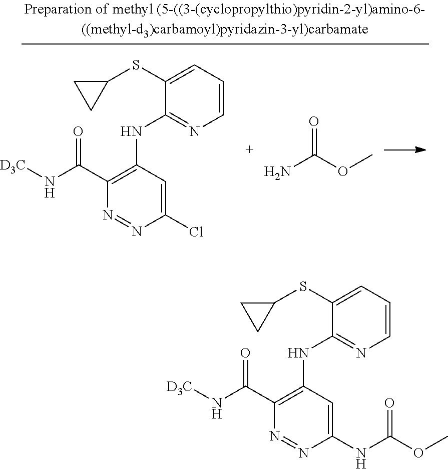

To a well stirred solution of 6-chloro-4-((3-(cyclopropylthio)pyridin-2-yl)amino)-N-(methyl-d₃)pyridazine-3-carboxamide (200 mg, 0.590 mmol) in 1,4-dioxane 8.0 mL) in a 20 mL sealed vial were added methyl carbamate (133 mg, 1.771 mmol) and Pd₂dba₃(27.0 mg, 0.030 mmol), DCPF (17.08 mg, 0.030 mmol) followed by Cs₂CO₃ (288 mg, 0.885 mmol). The reaction mixture was degassed with N₂ for 5 min. The resultant reaction mixture was heated to 110° C. for 3 h, monitoring by TLC. After completion of the reaction, the mixture was diluted with ethyl acetate (100 mL), filtered through a celite pad and the pad was thoroughly washed with ethyl acetate (100 mL). The filtrate was washed with water (2×50 mL) followed by brine (50 mL) and dried over Na₂SO₄. The solvent was evaporated under reduced pressure and the resultant crude residue was purified by reverse phase column (C18) using 45-50% water (0.1 ammonium acetate) in acetonitrile to give methyl (5-((3-(cyclopropylthio)pyridin-2-yl)amino)-6-((methyl-d₃)carbamoyl)pyridazin-3-yl)carbamate (50 mg, 0.123 mmol, 20.87% yield) as an off-white solid.

MS (M+1) m/z: 378 (M+H)+; LC retention time 2.397 min [Method A].

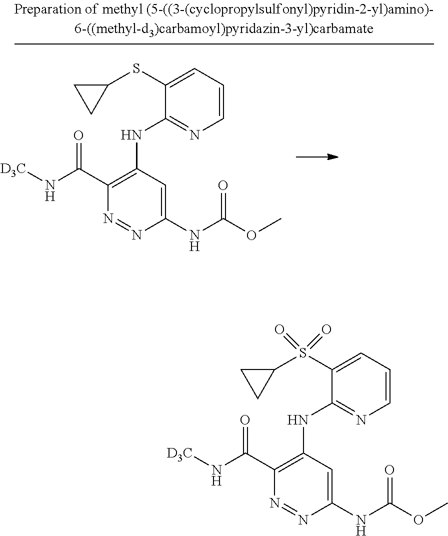 [SMILES: [2H]C([2H])([2H])NC(=O)c1nnc(NC(=O)OC)cc1Nc1ncccc1SC1CC1 ; [2H]C([2H])([2H])NC(=O)c1nnc(NC(=O)OC)cc1Nc1ncccc1S(=O)(=O)C1CC1]

To a well stirred solution of methyl (5-((3-(cyclopropylthio)pyridin-2-yl)amino)-6-((methyl-d₃)carbamoyl)pyridazin-3-yl)carbamate (50 mg, 0.132 mmol) in methanol (10 mL):water (1.0 mL) in a 25 mL two neck round bottom flask was added oxone (244 mg, 0.397 mmol). The resultant reaction mixture was stirred at room temperature for 24 h. After completion of the reaction, the mixture was diluted with DCM (50 mL), filtered through a celite pad and the pad was thoroughly washed with DCM (50 mL). The filtrate was washed with water (2×50 mL) followed by brine (50 mL) and dried over Na₂SO₄. The solvent was evaporated under reduced pressure and the resultant crude residue was purified by reverse phase column (C18) chromatography using 40-45% water (0.1% ammonium formate) in acetonitrile to give methyl (5-((3-(cyclopropylsulfonyl)pyridin-2-yl)amino)-6-((methyl-d₃)carbamoyl)pyridazin-3-yl)carbamate (15 mg, 0.034 mmol, 25.7% yield) as a off-white solid.

Analytical data:
HPLC-Purity: 92.883%. Method info: Column: Kinetex Biphenyl (100×4.6) mm,2.6μm
Mobile phase A: 0.05% TFA in water:ACN (95:5). Mobile phase B: ACN:0.05% TFA in water (95:5) Flow: 1.0 mL/min, RT 5.969 min).
LC-MS-Purity: 94.12%; m/z (E+): 410.2 [M+H]+. Method info: Column: Kinetex XB-C18 (75×3) mm, 2.6μm. Mobile Phase A: 5 mM Ammonium formate pH 3.3:ACN (98:02) Mobile Phase B: ACN: Buffer (98:02) Flow Rate: 1.0 ml/min, RT 1.296 min.

### ¹H-NMR (400 MHz, DMSO-d6): δ 12.12 (s, 1H), 10.96 (s, 1H), 9.27 (s, 1H), 9.17 (s, 1H), 8.64 (dd, J=1.60, 4.80 Hz, 1H), 8.25 (dd, J=2.00, 7.80 Hz, 1H), 7.35-7.32 (m, 1H), 3.72 (s, 3H), 3.15-3.08 (m, 1H), 1.21-1.17 (m, 2H), 1.09-1.05 (m, 2H).

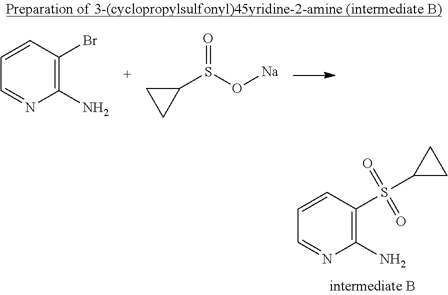

To a solution of 3-bromopyridin-2-amine (0.5 g, 2.89 mmol) in DMSO (10 mL) were added sodium cyclopropanesulfinate (0.481 g, 3.76 mmol), L-proline (0.067 g, 0.578 mmol), copper (I) iodide (0.110 g, 0.578 mmol) and sodium hydroxide (0.023 g, 0.578 mmol). The resultant mixture was degassed with N₂ for 5 min and heated to 120° C. for 12 h, monitoring by TLC. After completion of the reaction, the mixture was quenched with H₂O (100 mL), extracted with EtOAc (100 mL×3). The combined organic layers were concentrated under reduced pressure to give the crude product, which was purified by silica gel chromatography (PE/EtOAc= 2/1) to give 3-(cyclopropylsulfonyl)pyridin-2-amine (0.15 g, 0.681 mmol, 23.56% yield) as an off-white solid.

MS (M+1) m/z: 199 (M+H)+; LC retention time 0.827 min [Method A].

### EXAMPLE 3

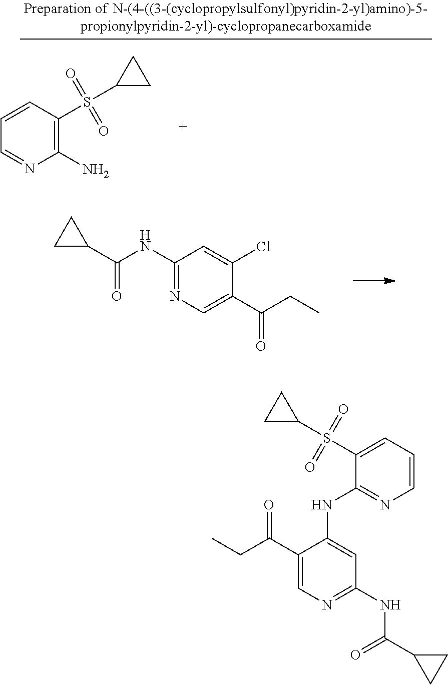 [SMILES: Nc1ncccc1S(=O)(=O)C1CC1 ; CCC(=O)c1cnc(NC(=O)C2CC2)cc1Cl ; CCC(=O)c1cnc(NC(=O)C2CC2)cc1Nc1ncccc1S(=O)(=O)C1CC1]

To a well stirred solution of 3-(cyclopropylsulfonyl)pyridin-2-amine (140 mg, 0.706 mmol) in 1,4-dioxane (8 mL) in a 20 mL sealed tube were added N-(4-chloro-5-propionylpyridin-2-yl)-cyclopropanecarboxamide (178 mg, 0.706 mmol) and BINAP (21.99 mg, 0.035 mmol), Pd₂(dba)₃ (32.3 mg, 0.035 mmol) followed by Cs₂CO₃ (460 mg, 1.412 mmol). The reaction mixture was degassed with N₂ for 5 min. The resultant mixture was heated to 110° C. for 3 h, monitoring by TLC. After completion of the reaction, the mixture was diluted with ethyl acetate (100 mL), filtered through a celite pad and the pad was thoroughly washed with ethyl acetate (100 mL). The filtrate was washed with water (2×50 mL) followed by brine (50 mL) and dried over Na₂SO₄. The solvent was evaporated off under concentrated under reduced pressure and the resultant crude residue was purified by prep HPLC purification to give N-(4-((3-(cyclopropylsulfonyl)pyridin-2-yl)amino)-5-propionylpyridin-2-yl)cyclopropanecarboxamide (75 mg, 0.179 mmol, 25.3% yield) as an off-white solid.

Analytical data:
HPLC-Purity: 98.894%. Method info: Column: Kinetex Biphenyl (100×4.6) mm, 2.6μm
Mobile phase A: 0.05% TFA in water:ACN (95:5). Mobile phase B: ACN:0.05% TFA in water (95:5) Flow: 1.0 mL/min, RT 7.320 min.
LC-MS-Purity: 93.35%; m/z (E+):415.2 [M+H]+. Method info: Column: Kinetex XB-C18 (75×3.0) mm, 2.6μm Mobile Phase A: 5 mM Ammonium formate pH 3.3:ACN (98:02) Mobile Phase B: ACN: Buffer (98:02) Flow Rate: 1.0 ml/min RT 2.179 min)
¹H-NMR (400 MHz, DMSO-d6): δ 11.89 (s, 1H), 10.98 (s, 1H), 8.98 (d, J=14.80 Hz, 2H), 8.60 (dd, J=2.00, 4.80 Hz, 1H), 8.24 (dd, J=1.60, 7.80 Hz, 1H), 7.34-7.31 (m, 1H), 3.14 (q, J=7.20 Hz, 2H), 3.00-2.96 (m, 1H), 2.08-2.02 (m, 1H), 1.15-0.11 (m, 4H), 1.08-1.04 (m, 4H), 0.96 (t, J=402.80 Hz, 3H).

### Preparation of Methyl (4-((3-(cyclopropylsulfonyl)pyridin-2-yl)amino)-5-propionylpyridin-2-yl)-carbamate

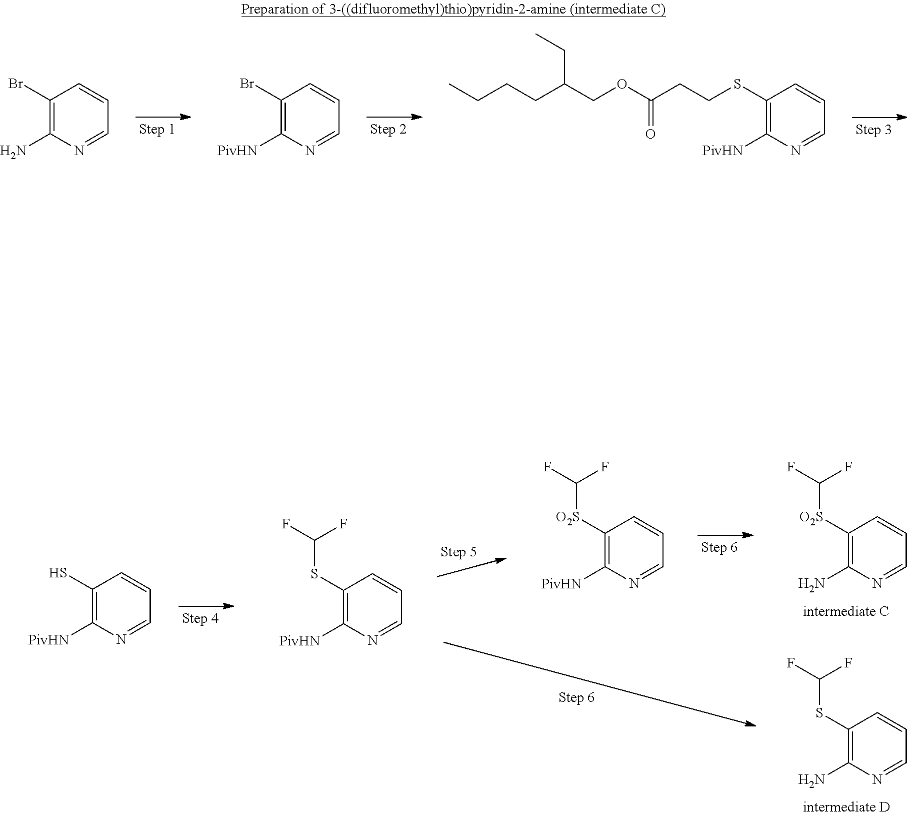

### Step 1

To a stirred solution of 3-bromopyridin-2-amine (10.0 g, 57.8 mmol) in DCM (100 mL) was added DIPEA (16.11 mL, 116 mmol) followed by pivaloyl chloride (8.53 mL, 69.4 mmol) at 0° C. Then the reaction mixture was stirred at room temperature for 2h. After completion of the reaction, the reaction mixture was diluted with water (100 mL) and then extracted with DCM (2×300 mL). The combined organic layer was washed with 10% aqueous NaHCO3 solution (100 mL), saturated aqueous brine solution (100 mL), dried over anhydrous Na₂SO₄, filtered and concentrated under vacuum to yield the crude product. The crude residue was purified by column chromatography (Biotage, SiO₂) eluted with 40-70% ethyl acetate in petroleum ether to give the desired product N-(3-bromopyridin-2-yl)pivalamide (15 g, 56.6 mmol, 98% yield) isolated as brown solid.

MS (M+1) m/z: 259 (M+H)+; LC retention time 1.277 min [Method D].

### ¹H-NMR (400 MHz, DMSO-d6): δ 9.72 (s, 1H), 8.44 (dd, J=1.60, 4.80 Hz, 1H), 8.13 (dd, J=1.60, 8.00 Hz, 1H), 7.26 (dd, J=4.80, 8.00 Hz, 1H), 1.23 (s, 9H).

### Step 2

3-Mercaptopropionic acid 2-ethylhexyl ester (7.64 g, 35.0 mmol) and DIPEA (15.28 mL, 88 mmol) were added to a mixture of N-(3-bromopyridin-2-yl)pivalamide (7.5 g, 29.2 mmol), Pd₂(dba)₃ (1.336 g, 1.458 mmol) and xantphos (1.688 g, 2.92 mmol) in 1,4-dioxane (100 mL) under N₂ atmosphere. The reaction was heated to 100° C. in a sealed tube for 16 h. The reaction mixture was filtered through a celite pad and the pad was washed with EtOAc (200 mL). The collected filtrate was then washed with H₂O (50 mL) and aqueous brine solution (50 mL). The organic layer was dried over anhydrous Na₂SO₄ and filtered. The solvent was removed under reduced pressure and the crude residue was purified by column chromatography (Biotage, SiO₂) eluted with 40-70% ethyl acetate in petroleum ether to afford 2-ethylhexyl 3-((2-pivalamidopyridin-3-yl)thio)propanoate (8.0 g, 20.07 mmol, 68.8% yield).

MS (M+1) m/z: 395.2 (M+H)+; LC retention time=1.80 min [Method D].

### Step 3

A 21% w/w ethanol solution of NaOEt (11.9 mL, 34.87 mmol) was added to a solution of 2-ethylhexyl 3-((2-pivalamidopyridin-3-yl)thio)propanoate (12.5 g, 31.7 mmol) in THF (30 mL) at 0° C. The reaction mixture was stirred under nitrogen for 30 min at room temperature. DCM (100 mL) was added and this was stirred for 5 minutes and then water (50 mL) was added. The water phase was collected and brought to ˜ pH 5 with acetic acid and extracted with ethyl acetate (2×100 mL). The pooled organic phase was washed with saturated aqueous brine solution (50 mL), dried over anhydrous Na₂SO₄, filtered and concentrated in vacuo to provide crude N-(3-mercaptopyridin-2-yl)pivalamide (5.5 g, 25.4 mmol, 80% yield). The crude product was used as such without further purification.

MS (M+1) m/z: 211.0 (M+H)+; LC retention time=0.739 min [Method D].

### Step 4

KOH (53.4 g, 951 mmol) was added portion wise to water (100 mL) and the solution was cooled to 0° C. Acetonitrile (100 mL) and N-(3-mercaptopyridin-2-yl)pivalamide (10.0 g, 47.6 mmol) were added to the above reaction mixture. Diethyl (bromodifluoromethyl)-phosphonate (16.90 mL, 95 mmol) was added and the reaction mixture was allowed to reach room temperature and stirred at this temperature for 16 h. The reaction mixture was diluted with Et₂O (500 mL), and then washed with H₂O (50 mL) followed by saturated aqueous brine solution (50 mL). The collected organic layer was dried over Na₂SO₄, filtered, and the solvent was removed under reduced pressure to yield crude product. The crude residue was purified by column chromatography (Biotage, SiO₂) eluted with 20-50% ethyl acetate in petroleum ether to obtain N-(3-((difluoromethyl)thio)pyridin-2-yl)pivalamide (6.6 g, 25.4 mmol, 53.3% yield).

MS (M+1) m/z: 261.2 (M+H)+; LC retention time 1.171 min [Method D].

### ¹H-NMR (400 MHz, DMSO-d6): δ 10.08 (s, 1H), 8.49 (dd, J=2.00, 4.80 Hz, 1H), 8.06 (dd, J=1.60, 7.80 Hz, 1H), 7.53-7.26 (m, 2H), 1.23 (s, 9H).

### Step 5

To a stirred solution of N-(3-((difluoromethyl)thio)pyridin-2-yl)pivalamide (1.8 g, 6.92 mmol) in chloroform (25 mL), acetonitrile (25 mL) and water (37.5 mL) was added ruthenium (III) chloride trihydrate (0.362 g, 1.383 mmol) and sodium metaperiodate (11.83 g, 55.3 mmol) at 0° C. The resulting reaction mixture was stirred at room temperature for 3 h. The reaction mixture was quenched with aqueous saturated sodium bicarbonate solution. Then it was extracted with DCM (100 mL). The collected organic layer was dried over Na₂SO₄, filtered, and the solvent was removed under reduced pressure to yield crude product. The crude product was used as such without further purification.

MS (M+1) m/z: 293.0 (M+H)+; LC retention time 1.12 min [Method D].

### Step 6: Intermediate C

N-(3-((difluoromethyl)sulfonyl)pyridin-2-yl)pivalamide (8.1 g, 27.7 mmol) was taken up in 4 N aqueous HCl (41.6 ml, 166 mmol) and the reaction mixture was stirred for 3 h at 80° C. After completion, the reaction mixture was cooled in an ice bath and neutralized with solid NaHCO₃, the aqueous phase was extracted with DCM (2×100 mL) followed by CHCl3 (100 mL) and the combined organic phase was dried over anhydrous Na₂SO₄. After filtration, the solvent was removed under reduced pressure. The crude product was triturated using petroleum ether and then using a small amount of EtOAc. The product was further purified by RP column purification to obtain 3-((difluoromethyl)sulfonyl)pyridin-2-amine (3.8 g, 15.15 mmol, 54.7% yield) as white solid.

MS (M+1) m/z: 209.0 (M+H)+; LC retention time 0.88 min [Method D].

### ¹H-NMR (DMSO-d6): δ 8.39 (dd, J=4.8 &1.8 Hz, 1H), 7.90 (dd, J=8 & 1.6 Hz, 1H), 7.23 (t, J=52Hz, 1H), 7.05 (bs, 2H), 6.81 (dd, J=8 7 4.8 Hz, 1H).

### Step 6: Intermediate D

To a solution of N-(3-((difluoromethyl)thio)pyridin-2-yl)pivalamide (4.5 g, 17.29 mmol) in a 100 mL three neck round bottom flask was added 2 N HCl (51.9 mL, 104 mmol) and the resultant reaction mixture was stirred at 100° C. for 16 h. After completion, the reaction mixture was cooled to room temperature. The reaction mixture was diluted with water (100 mL), extracted with ethyl acetate (200 mL) and the aqueous layer was neutralised with saturated aqueous sodium bicarbonate solution (200 mL) and extracted with DCM (2×200 mL). The combined organic layer was dried over anhydrous Na₂SO₄ and concentrated under reduced pressure to give 3-((difluoromethyl)thio)pyridin-2-amine (2.5 g, 13.85 mmol, 80% yield) as a brown solid.

MS (M+1) m/z: 177.0 (M+H)+; LC retention time=0.528 min [Method D].

### EXAMPLE 4

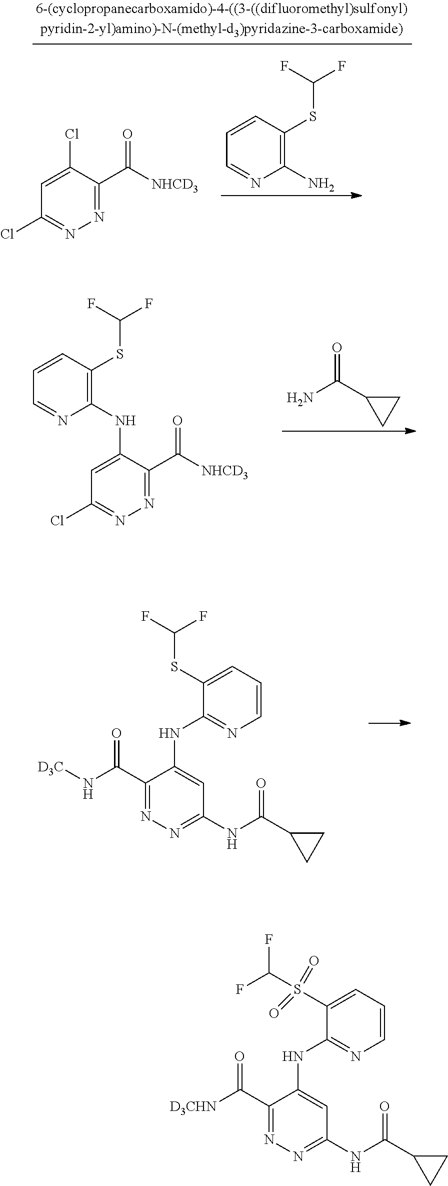

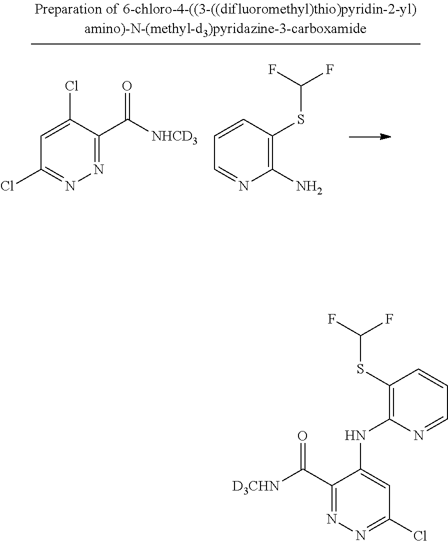

To a stirred solution of 3-((difluoromethyl)thio)pyridin-2-amine (2.5 g, 14.19 mmol) and 4,6-dichloro-N-(methyl-d₃)pyridazine-3-carboxamide (7.42 g, 35.5 mmol) in THF (25 mL) was added LiHMDS (35.5 mL, 35.5 mmol, 1 M solution in THF) dropwise over 10 minutes. The reaction mixture was stirred at room temperature for 30 minutes. The reaction mixture was quenched with water (250 mL) and stirred for 10 minutes. The resultant reaction mixture was filtered and the obtained solid compound was washed with n-pentane: diethylether (250 mL, 5:1) followed by drying gave desired 6-chloro-4-((3-((difluoromethyl)thio)pyridin-2-yl)amino)-N-(methyl-d₃)pyridazine-3-carboxamide (3 g, 8.53 mmol, 60% yield) as an off-white solid.

MS (M+1) m/z: 349.0 (M+H)+; LC retention time=2.69 min [Method A].

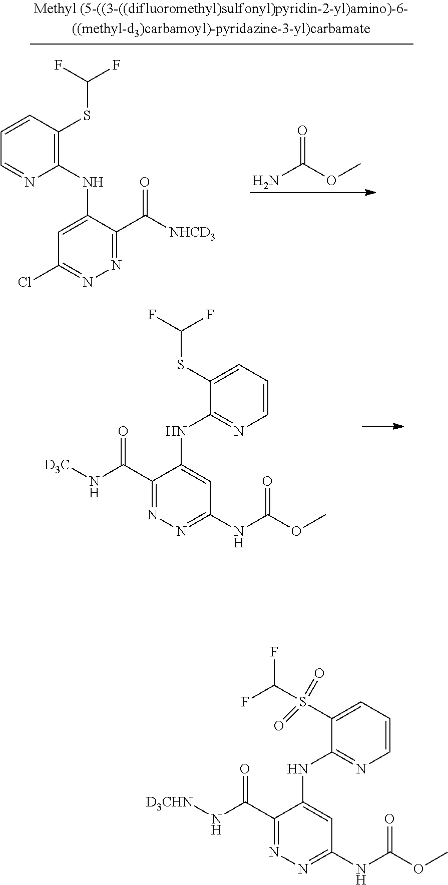

To the solution of 6-chloro-4-((3-((difluoromethyl)thio)pyridin-2-yl)amino)-N-(methyl-d₃)pyridazine-3-carboxamide (25 mg, 0.072 mmol) in dioxane (2 mL) was added cyclopropanecarboxamide (6.71 mg, 0.079 mmol), Pd₂(dba)₃ (3.28 mg, 3.58 μmol), 1,1′-bis(dicyclohexylphosphino)ferrocene (4.15 mg, 7.17 μmol) and cesium carbonate (70.1 mg, 0.215 mmol). The reaction mixture was degassed under N₂ for 5 mins. The reaction mixture was sealed and stirred in a pressure release vial at 120° C. for 1.5 h. The reaction solution was filtered through a celite pad and washed with 10% methanol in DCM (20 mL) and the filtrate was concentrated under reduced pressure. The crude product was triturated with diethylether to obtain crude product. This was further purified by crystallization in MeOH and hexane. The desired product 6-(cyclopropanecarboxamido)-4-((3-((difluoromethyl)thio)pyridin-2-yl)amino)-N-(methyl-d₃)pyridazine-3-carboxamide (25 mg, 0.027 mmol, 37.0% yield) was isolated as brown solid.

### LC-MS Analysis:

### Column: Aquity Uplc BEH C18 (50×3.0) mm, 1.7μm

### Mobile Phase A: 0.1% TFA in Water

### Mobile Phase B: 0.1% TFA in ACN

Flow Rate: 1.0 ml/min.
Desired Product mass m/z: 398.0 (M+H)+. LC retention time 1.57 min.

### Preparation of 6-(cyclopropanecarboxamido)-4-((3-

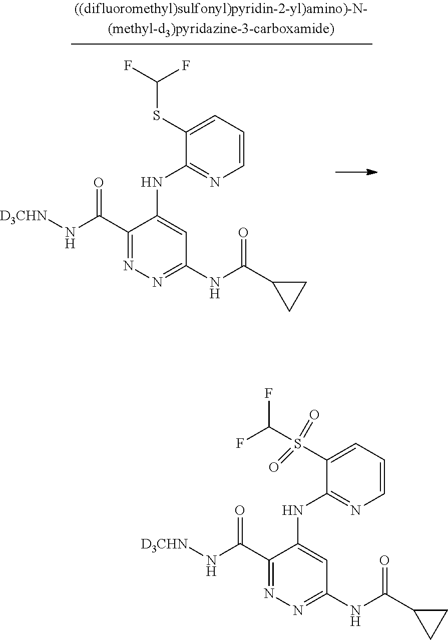

A solution of 6-(cyclopropanecarboxamido)-4-((3-((difluoromethyl)thio)pyridin-2-yl)amino)-N-(methyl-d₃)pyridazine-3-carboxamide (0.25 g, 0.629 mmol) in acetic acid (5 mL) was heated to 55° C. and stirred for 5 min, then sodium tungstate dihydrate (0.415 g, 1.258 mmol) and H₂O₂ (0.386 mL, 12.58 mmol) were added at 55° C. The reacton mixture was stirred for 2 hr at 55° C. After completion of the reaction, it was diluted with ice water and bacified with sat. NH₄CO₃ to pH 7-8 and then extrated with 10% MeOH in DCM (150 mL). The combined organic layer was washed with brine (10 mL), dried over Na₂SO₄, filtered, and evaporated to give 40 mg of crude product. The crude material was dissolved in 5 mL of dioxane and 100 mg of bis pinacolato diboron was added and the mixture stirred at 100° C. for 2h. After completion of the reaction the mixture was concentrated and n-hexane (3 mL) added. The solid that formed was filtered off and purified by reverse phase prep HPLC to yield the desired product 6-(cyclopropanecarboxamido)-4-((3-((difluoromethyl)sulfonyl)-pyridin-2-yl) amino)-N-(methyl-d₃)pyridazine-3-carboxamide, as TFA salt (30 mg, 0.050 mmol, 7.96% yield).

HPLC-01: Method information

### Column: Kinetex Biphenyl (100×4.6) mm, 2.6μm

Mobile phase A: 0.05% TFA in water:ACN (95:5)
Mobile phase B: ACN:0.05% TFA in water (95:5)
Flow: 1.0 mL/min.

### RT=7.413, Purity=91%

HPLC-02: Method information

### Column: Kinetex EVO C18 (100×4.6) mm, 2.6μm

Mobile phase A: 0.05% TFA in water:ACN (95:5)
Mobile phase B: ACN:0.05% TFA in water (95:5)
Flow: 1.0 mL/min

### RT=6.558, Purity=94%

LCMS: Method information:

### Column: Kinetex XB-C18 (75×3.0) mm, 2.6μm

Mobile Phase A: 5 mM Ammonium formate pH 3.3:ACN (98:02)

### Mobile Phase B: ACN:Buffer (98:02)

Flow Rate: 1.0 ml/min.
Desired Product mass m/z=430.0 (M+1); Retention time=1.646 min.

### ¹H-NMR (400 MHz, DMSO-d6): δ 12.29 (s, 1H), 11.48 (s, 1H), 9.43 (s, 1H), 9.19 (s, 1H), 8.73-8.75 (m, 1H), 8.73-8.75 (m, 1H), 7.27-7.41 (m, 2H), 7.27-7.41 (m, 1H), 0.86-0.88 (m, 4H),

### EXAMPLE 5

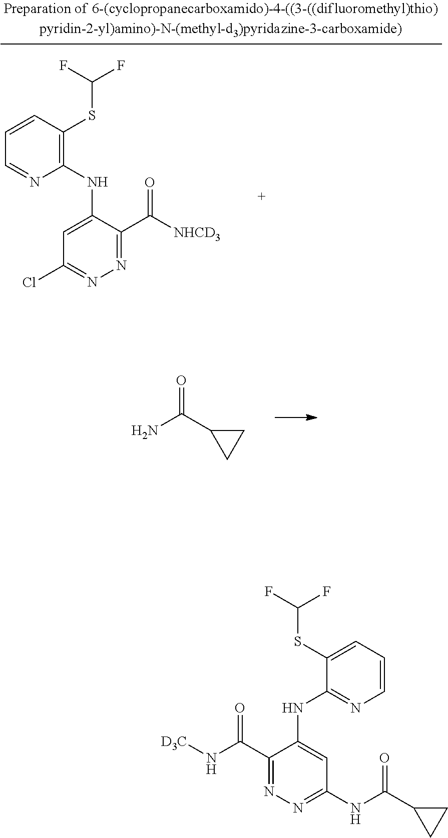

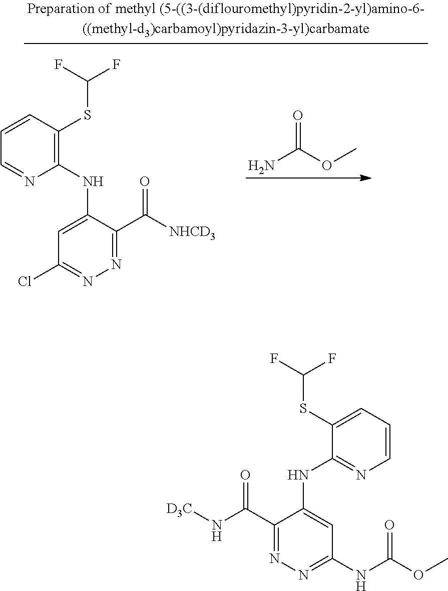

To a solution of 6-chloro-4-((3-((difluoromethyl)thio)pyridin-2-yl)amino)-N-(methyl-d₃)pyridazine-3-carboxamide (300 mg, 0.860 mmol) in dioxane (10 mL) was added methyl carbamate (646 mg, 8.60 mmol), Pd₂(dba)₃ (39.4 mg, 0.043 mmol), 1,1′-bis(dicyclohexylphosphino)ferrocene (49.8 mg, 0.086 mmol) and cesium carbonate (841 mg, 2.58 mmol). The reaction mixture was degassed under N₂ for 5 mins. The sealed reaction mixture was stirred at 120° C. for 2 h. The reaction solution was filtered through a celite pad, washed with 10% methanol in DCM (100 mL) and concentrated under reduced pressure. The crude product was washed with diethyl ether (50 mL) to remove ligand byproducts. Desired product methyl (5-((3-((difluoromethyl)thio)pyridin-2-yl)amino)-6-((methyl-d₃)carbamoyl)pyridazin-3-yl)carbamate (200 mg, 0.516 mmol, 60.0% yield) was isolated as brown solid.

Desired product mass m/z: 388.0 (M+1)+. Retention time 1.81 min.
LC-MS Analysis: Method information:

### Column: Kinetex XB-C18 (75×3.0) mm, 2.6μm

Mobile Phase A: 5 mM Ammonium formate pH 3.3:ACN (98:02)

### Mobile Phase B: ACN: Buffer (98:02)

Flow Rate: 1.0 ml/min.

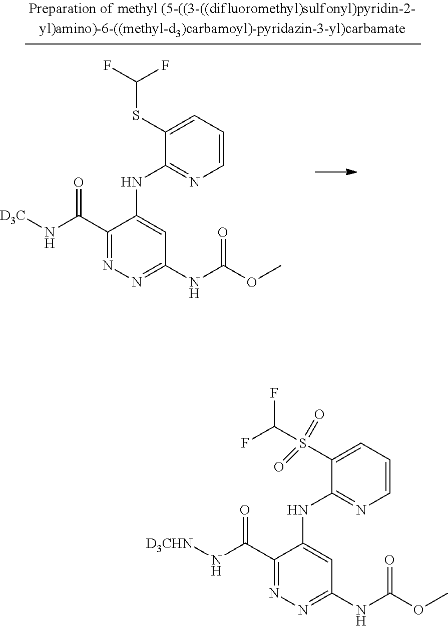

A stirred solution of methyl (5-((3-((difluoromethyl)thio)pyridin-2-yl)amino)-6-((methyl-d₃)carbamoyl)pyridazin-3-yl)carbamate (0.2 g, 0.516 mmol) in acetic acid (5 mL) was heated to 55° C. and stirred for 5 min, then sodium tungstate dihydrate (0.341 g, 1.033 mmol) and H₂O₂ (1.055 mL, 10.33 mmol) were added. The reacton mixture was stirred for 2 hr at 55° C. After completion of the reaction the mixture was diluted with ice water and bacified with sat. NH₄CO₃ to PH- 7-8 and extracted with 10% MeOH in DCM (150 mL). The combined organic layer was washed with brine (10 mL), dried over Na₂SO₄, filtered, and evaporated to give 40 mg of crude product. The crude product was dissolved in 5 mL of dioxane and 100 mg of bis pinacolato diboron was added and the mixture was stirred at 100° C. for 2 h. Upon completion the reaction was concentrated and n-hexane (3 mL) added, the solid was filtered and purified by reverse phase prep HPLC to yield the desired product methyl (5-((3-((difluoromethyl)-sulfonyl)pyridin-2-yl)amino)-6-((methyl-d₃)carbamoyl)-pyridazin-3-yl)carbamate as TFA salt (7.3 mg, 0.013 mmol, 2.509% yield).

HPLC-01: Method information

### Column: Kinetex Biphenyl (100×4.6) mm, 2.6μm

Mobile phase A: 0.05% TFA in water:ACN (95:5)
Mobile phase B: ACN:0.05% TFA in water (95:5)
Flow: 1.0 mL/min.

### RT=6.896, Purity=96.65%

HPLC-02: Method information

### Column: Kinetex EVO C18 (100×4.6) mm, 2.6μm

Mobile phase A: 0.05% TFA in water:ACN (95:5)
Mobile phase B: ACN:0.05% TFA in water (95:5)
Flow: 1.0 mL/min

### RT=6.035, Purity=94.6%

LCMS: Method information:

### Column: Kinetex XB-C18 (75×3.0) mm, 2.6μm

Mobile Phase A: 5 mM Ammonium formate pH 3.3:ACN (98:02)

### Mobile Phase B: ACN:Buffer (98:02)

Flow Rate: 1.0 ml/min.
Desired Product mass m/z=420.0 (M+1); Retention time=1.459 min.

### ¹H-NMR (400 MHz, DMSO-d6): δ 12.31 (s, 1H), 10.99 (s, 1H), 9.27 (s, 1H), 9.17 (s, 1H), 8.76-8.78 (m, 1H), 8.36-8.38 (m, 1H), 7.28-7.54 (m, 2H), 3.72 (s, 3H).

### EXAMPLE 6

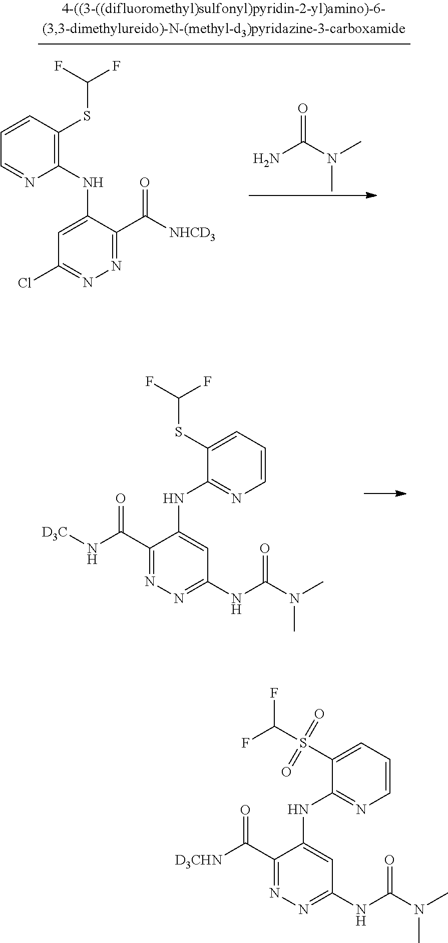

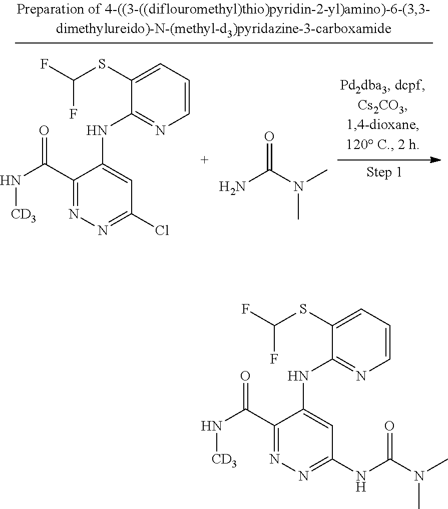

To a stirred solution of 1,1-dimethylurea (168 mg, 1.911 mmol) and 6-chloro-4-((3-((difluoromethyl)thio)pyridin-2-yl)amino)-N-(methyl-d₃)pyridazine-3-carboxamide (400 mg, 1.147 mmol) in 1,4-dioxane (10 mL), were added Cs₂CO₃ (934 mg, 2.87 mmol), Pd₂dba₃ (88 mg, 0.096 mmol), 1,l′-bis(dicyclohexylphosphino)ferrocene (60.3 mg, 0.096 mmol) and the mixture purged with N₂ for 5 mins. The reaction mixture was sealed and stirred at 120° C. for 2h. The reaction mixture was filtered through a celite pad, washed with ethyl acetate (100 mL) and concentrated under reduced pressure. The crude residue was purified by reverse phase column chromatography to obtain the desired product 4-((3-((difluoromethyl)-thio)pyridin-2-yl)amino)-6-(3,3-dimethylureido)-N-(methyl-d₃)pyridazine-3-carboxamide (160 mg, 0.276 mmol, 28.8% yield) as brown solid.

MS (M+1) m/z: 401.0 (M+H)⁺; LC retention time=0.845 min [Method D].

The following Intermediates were prepared in a similar manner.

| | | | m/z | Rt (min) |
|---|---|---|---|---|
| Intermediates | R | MW | [M + H]⁺ | [Method] |
| 7 | 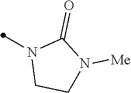 | 412.43 | 413.0 | 0.94 [C] |
| 8 | 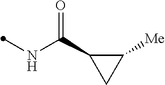 | 411.45 | 412.0 | 2.20 [D] |
| 9 | 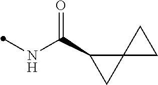 | 423.46 | 424.0 | 2.21 [C] |
| 10 | 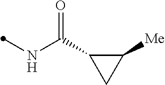 | 411.45 | 412.0 | 2.18 [D] |
| 11 | 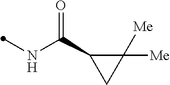 | 425.47 | 426.0 | 2.47 [D] |
| 12 | 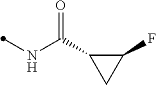 | 415.41 | 416.0 | 2.06 [D] |
| 13 | 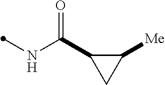 | 411.45 | 412.1 | 2.40 [C] |
| 14 | 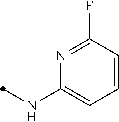 | 424.42 | 425.1 | 2.33 [C] |

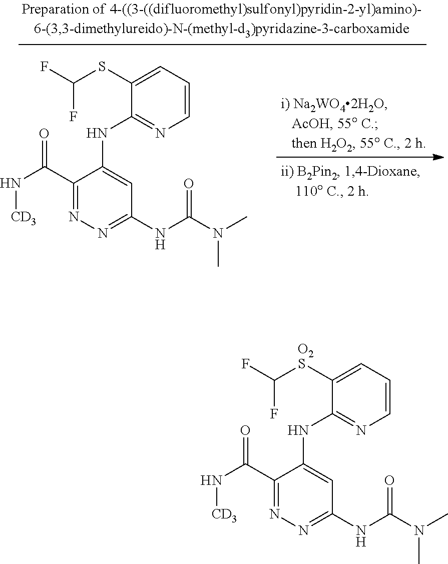 [SMILES: [2H]C([2H])([2H])NC(=O)c1nnc(NC(=O)N(C)C)cc1Nc1ncccc1SC(F)F ; [2H]C([2H])([2H])NC(=O)c1nnc(NC(=O)N(C)C)cc1Nc1ncccc1S(=O)(=O)C(F)F]

A stirred solution of 4-((3-((difluoromethyl)thio)pyridin-2-yl)amino)-6-(3,3-dimethylureido)-N-(methyl-d₃)pyridazine-3-carboxamide (160 mg, 0.400 mmol) and sodium tungstate dihydrate (264 mg, 0.799 mmol) in acetic acid (5 mL) was heated to 55° C. and stirred for 5 min, then added H₂O₂ (0.816 mL, 7.99 mmol) at 55° C. The reaction mixture was stirred at 55° C. for 2h. After completion, the reaction mixture was diluted with ice water, basified with saturated aqueous NaHCO₃ solution up to pH 7˜8 and extracted with 10% MeOH in DCM (150 mL). The combined organic layer was washed with brine (20 mL), dried over anhydrous Na₂SO₄, filtered, and concentrated under reduced pressure to obtain the crude product. The crude residue was dissolved in 1,4-dioxane (5 mL) and 100 mg of bispin was added and the mixture was stirred at 100° C. for 2 h. After completion, the solvent was evaporated and the crude product was purified by reverse phase pep-HPLC (using 0.1% TFA in ACN) to obtain desired product 4-((3-((difluoromethyl)sulfonyl)pyridin-2-yl)amino)-6-(3,3-dimethylureido)-N-(methyl-d₃)pyridazine-3-carboxamide as TFA salt (9.1 mg, 0.015 mmol, 3.81% yield) as pale yellow solid.

MS (M+1) m/z: 433.0 (M+H)+; LC retention time=1.26 min [Method A].

### ¹H-NMR (400 MHz, DMSO-d6): δ 12.21 (s, 1H), 9.63 (s, 1H), 9.08 (s, 2H), 8.75-8.73 (m, 1H), 8.36-8.33 (m, 1H), 7.52-7.36 (m, 2H), 2.97 (s, 6H).

The following example (7) was prepared in a similar manner to the preparation of example 6.

| | | | m/z | Rt (min) |
|---|---|---|---|---|
| example | R | MW | [M + H]⁺ | Method] |
| 7 | 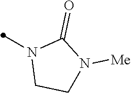 | 444.43 | 445.1 | 2.13 [B] |

### EXAMPLE 8

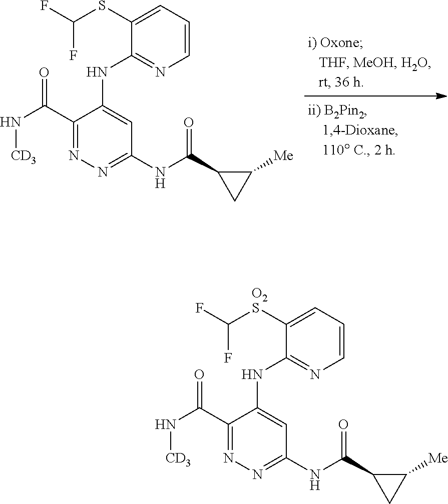 [SMILES: [2H]C([2H])([2H])NC(=O)c1nnc(NC(=O)[C@@H]2C[C@H]2C)cc1Nc1ncccc1SC(F)F ; [2H]C([2H])([2H])NC(=O)c1nnc(NC(=O)[C@@H]2C[C@H]2C)cc1Nc1ncccc1S(=O)(=O)C(F)F]

To a stirred solution of 4-((3-((difluoromethyl)thio)pyridin-2-yl)amino)-N-(methyl-d₃)-6-((1R,2R)-2-methylcyclopropane-1-carboxamido)pyridazine-3-carboxamide (0.120 g, 0.292 mmol) in THF (10 mL):methanol (10 mL):water (2 mL) was added oxone (0.896 g, 1.458 mmol). The resultant reaction mixture was stirred at room temperature for 36 h. After completion, the reaction mixture was diluted with DCM (100 mL), filtered through a celite pad and washed with DCM (100 mL). The filtrate was washed with water (2×100 mL) followed by saturated aqueous brine (100 mL) and dried over anhydrous Na₂SO₄. Solvent was evaporated under reduced pressure. To the resultant crude residue (140 mg) in 1,4-dixane (10 mL) was added bispin (0.148 g, 0.583 mmol) and stirred at 100° C. for 2 h. Then the reaction mixture was concentrated under reduced pressure and the obtained crude compound was purified by reverse phase prep-HPLC (0.1% ammonium formate in ACN) to give 4-((3-((difluoromethyl)sulfonyl)pyridin-2-yl)amino)-N-(methyl-d₃)-6-((1R,2R)-2-methylcyclopropane-1-carboxamido)pyridazine-3-carboxamide (12 mg, 0.027 mmol, 9.13% yield) as a yellow solid.

MS (M+1) m/z: 441.1 (M+H)+; LC retention time=2.33 min [Method B].

### ¹H-NMR (400 MHz, DMSO-d6): δ 12.28 (s, 1H), 11.40 (s, 1H), 9.40 (s, 1H), 9.19 (s, 1H), 8.74-8.73 (m, 1H), 8.37-8.34 (m, 1H), 7.52-7.27 (m, 2H), 1.90-1.86 (m, 1H), 1.32-1.27 (m, 1H), 1.11-1.04 (m, 4H), 0.75-0.71 (m, 1H).

| Example | | | m/z | Rt (min) |
|---|---|---|---|---|
| (9-14) | R | MW | [M + H]⁺ | [Method] |
| 9 | 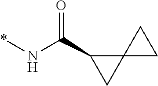 [SMILES: *NC(=O)[C@@H]1CC12CC2] | 455.46 | 456.1 | 2.36 [B] |
| 10 | 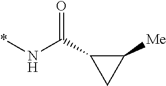 | 443.44 | 444.2 | 2.25 [B] |
| 11 | 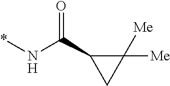 [SMILES: *NC(=O)[C@@H]1CC1(C)C] | 457.47 | 458.2 | 2.56 [A] |
| 12 | 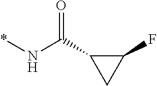 [SMILES: *NC(=O)[C@H]1C[C@@H]1F] | 447.41 | 448.1 | 2.31 [B] |
| 13 | 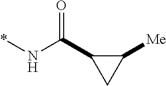 | 443.44 | 444.1 | 2.11 [B]* |
| 14 | 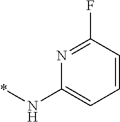 [SMILES: *Nc1cccc(F)n1] | 456.42 | 457.1 | 2.24 [B] |

* The isomers separated using chiral SFC.

| Example | ¹H NMR |
|---|---|
| 7 | ¹H-NMR (400 MHz, DMSO-d₆): δ 12.33 (s, 1H), 9.54 (s, 1H), 9.28 (s, |
| | 1H), 8.73 (d, J = 3.20 Hz, 1H), 8.36 (d, J = 6.40 Hz, 1H), 7.53-7.24 (m, |
| | 2H), 4.55-4.51 (m, 2H), 4.36-4.32 (m, 2H). |
| 8 | ¹H-NMR (400 MHz, DMSO-d₆): δ 12.20 (s, 1H), 9.57 (s, 1H), 9.17 (d, |
| | J = 4.80 Hz, 1H), 8.74-8.72 (m, 1H), 8.36-8.33 (m, 1H), 7.51-7.36 (m, |
| | 2H), 4.13-4.09 (m, 2H), 3.42-3.37 (m, 2H), 2.80 (s, 3H). |
| 9 | ¹H-NMR (400 MHz, DMSO-d₆): δ 12.28 (s, 1H), 11.28 (s, 1H), 9.43 (s, |
| | 1H), 9.18 (s, 1H), 8.75 (s, 1H), 8.38 (s, 1H), 7.52-7.26 (m, 2H), 1.43- |
| | 1.38 (m, 2H), 0.91-0.84 (m, 3H), 0.79-0.78 (m, 1H). |
| 10 | ¹H-NMR (400 MHz, DMSO-d₆): δ 12.28 (s, 1H), 11.40 (s, 1H), 9.40 (s, |
| | 1H), 9.19 (s, 1H), 8.74-8.73 (m, 1H), 8.37-8.34 (m, 1H), 7.52-7.27 (m, |
| | 2H), 1.90-1.86 (m, 1H), 1.32-1.27 (m, 1H), 1.13-1.05 (m, 4H), 0.75-0.72 |
| | (m, 1H). |
| 11 | ¹H-NMR (400 MHz, DMSO-d₆): δ 12.28 (s, 1H), 11.31 (s, 1H), 9.42 (s, |
| | 1H), 9.19 (s, 1H), 8.74-8.73 (m, 1H), 8.36-8.34 (m, 1H), 7.52-7.26 (m, |
| | 2H), 2.01-1.98 (m, 1H), 1.17 (s, 3H), 1.14 (s, 3H), 1.04-1.01 (m, 1H), |
| | 0.88-0.85 (m, 1H). |
| 12 | ¹H-NMR (400 MHz, DMSO-d₆): δ 12.32 (s, 1H), 11.64 (s, 1H), 9.41 (s, |
| | 1H), 9.22 (s, 1H), 8.73-8.72 (m, 1H), 8.37-8.35 (m, 1H), 7.53-7.27 (m, |
| | 2H), 5.02-4.84 (m, 1H), 1.64-1.60 (m, 1H), 1.59-1.54 (m, 2H). |
| 13 | ¹H-NMR (400 MHz, DMSO-d₆): δ 12.29 (s, 1H), 11.38 (s, 1H), 9.43 (s, |
| | 1H), 9.19 (s, 1H), 8.75-8.73 (m, 1H), 8.37-8.35 (m, 1H), 7.41-7.08 (t, J = |
| | 58.40 Hz, 2H), 2.15-2.11 (m, 1H), 1.36-1.33 (m, 1H), 1.12 (d, J = 6.40 |
| | Hz, 3H), 1.05-1.00 (m, 1H), 0.85-0.81 (m, 1H). |
| 14 | ¹H-NMR (400 MHz, DMSO-d₆): δ 12.37 (s, 1H), 10.63 (s, 1H), 9.51 (s, |
| | 1H), 9.16 (s, 1H), 8.77-8.75 (m, 1H), 8.36 (dd, J = 2.00, 8.00 Hz, 1H), |
| | 7.94-7.88 (m, 1H), 7.58 (dd, J = 2.00, 8.00 Hz, 1H), 7.54-7.28 (m, 2H), |
| | 6.69 (dd, J = 2.40, 8.00 Hz, 1H). |

### EXAMPLE 15

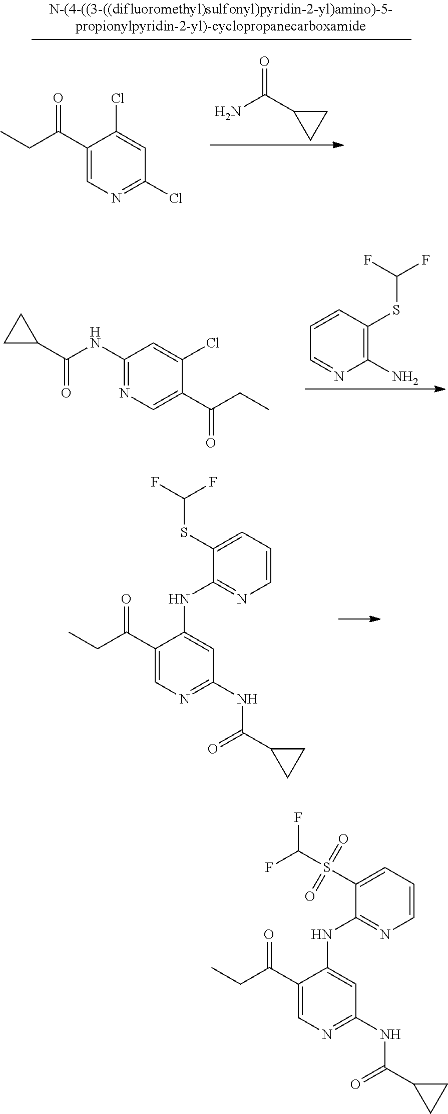

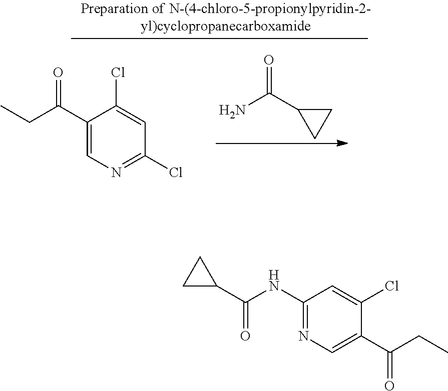 [SMILES: CCC(=O)c1cnc(Cl)cc1Cl ; NC(=O)C1CC1 ; CCC(=O)c1cnc(NC(=O)C2CC2)cc1Cl]

To a stirred solution of 1-(4,6-dichloropyridin-3-yl)propan-1-one (300 mg, 1.470 mmol) in 1,4-dioxane (5 mL), in a microwave vial was added Cs₂CO₃ (1437 mg, 4.41 mmol), and cyclopropanecarboxamide (125 mg, 1.470 mmol). The reaction mixture was degassed for 10 min, followed by addition of 1,1-bis(dicyclohexylphosphino)ferrocene (1021 mg, 1.764 mmol), Pd₂(dba)₃ (269 mg, 0.294 mmol) and degassing continued for another 2 min. Then the reaction mixture was sealed and stirred at 80° C. for 2 h. The reaction mixture was filtered through a syringe pad then transferred into water and extracted with ethyl acetate. The combined organic layer was dried over sodium sulfate and concentrated under reduced pressure. The crude compound was purified by flash column chromatography, eluted in 10-12% ethyl acetate in hexane to get N-(4-chloro-5-propionylpyridin-2-yl)cyclopropane-carboxamide (150 mg, 0.522 mmol, 35.5% yield) as a pale yellow solid.

### LCMS: MS: M/z:[M+1]: 253

### Column: Kinetex XB-C18 (75×3.0) mm, 2.6μm

Mobile Phase A: 5 mM Ammonium formate pH 3.3:ACN (98:02)

### Mobile Phase B: ACN:Buffer (98:02)

Flow Rate: 1.0 ml/min, RT: 2.236.

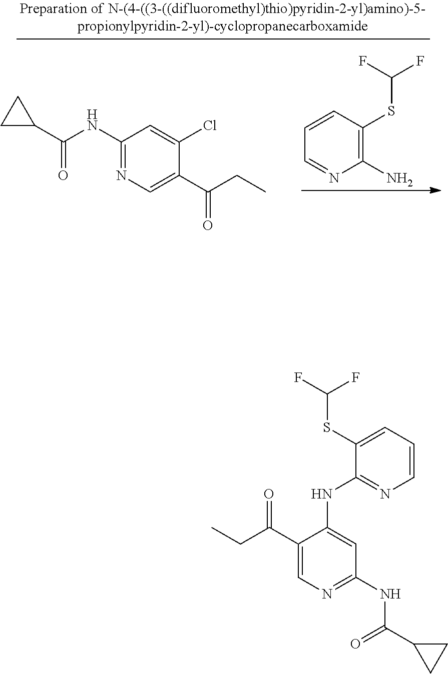 [SMILES: CCC(=O)c1cnc(NC(=O)C2CC2)cc1Cl ; Nc1ncccc1SC(F)F ; CCC(=O)c1cnc(NC(=O)C2CC2)cc1Nc1ncccc1SC(F)F]

To a stirred solution of N-(4-chloro-5-propionylpyridin-2-yl)cyclopropanecarboxamide (430 mg, 1.703 mmol),3-((difluoromethyl)thio)pyridin-2-amine (300 mg, 1.703 mmol) in 1,4-dioxane (5 mL), in a 20 mL reaction vial, Cs₂CO₃ (1110 mg, 3.41 mmol), Pd₂(dba)₃ (78 mg, 0.085 mmol), 1,l′-bis(dicyclohexyl-phosphino)ferrocene (99 mg, 0.170 mmol) were added under continious N₂ purging. The reaction mixture was stirred for 2 h at 120° C., monitoring by TLC. After consumption of starting material the crude product was filtered through a celite bed, washed with ethyl acetate (25 mL) and concentrated under reduced pressure. The residue was purified by column chromatography (Biotage, SiO₂, eluted with 5-25% ethyl acetate in petroleum ether) to give 200 mg of brown solid. The desired product methyl (4-((3-((difluoromethyl)thio)pyridin-2-yl)amino)-5-propionylpyridin-2-yl)carbamate (200 mg, 0.272 mmol, 33.0% yield) was obtained.

Desired Product mass m/z=393.1 (M+1); Retention time=2.251 min.
Method information: Column: Kinetex XB C18 2.6μm, (75×3) mm
Mobile phase: 5 mM ammonium formate (pH 3.3 using formic acid) in water
Mobile phase A: Buffer:Acetonitrile (98:02)
Mobile phase B: Acetonitrile:Buffer (98:02)
Flow rate: 1.0 mL/min

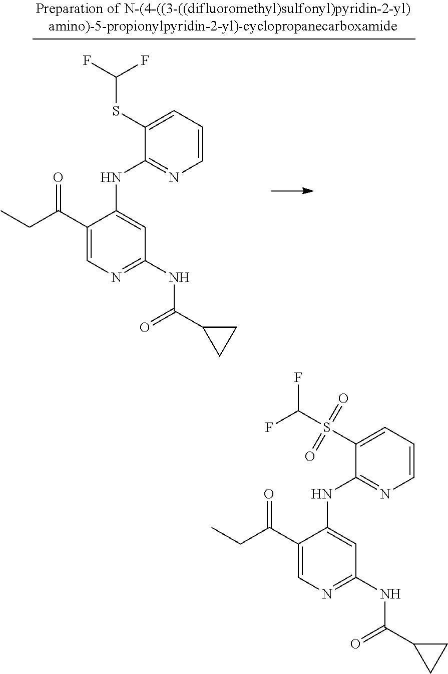 [SMILES: CCC(=O)c1cnc(NC(=O)C2CC2)cc1Nc1ncccc1SC(F)F ; CCC(=O)c1cnc(NC(=O)C2CC2)cc1Nc1ncccc1S(=O)(=O)C(F)F]

A stirred solution of N-(5-((3-((difluoromethyl)thio)pyridin-2-yl)amino)-6-propionylpyridin-3-yl)cyclopropanecarboxamide (0.2 g, 0.510 mmol) in acetic acid (5 mL) was heated to 55° C. and stirred for 5 min, then sodium tungstate dihydrate (0.336 g, 1.019 mmol) and H₂O₂ (1.041 mL, 10.19 mmol) were added. The reacton mixture was stirred for 2 hr at 55° C. After completion of the reaction, the mixture was diluted with ice water and adjusted with sat. NH₄CO₃ to PH- 7-8 and then extrated with 10% MeOH in DCM (150 mL). The combined organic layer was washed with brine (10 mL), dried over Na₂SO₄, filtered, and evaporated to give 41 mg of crude product. The crude was dissolved in 5 mL of dioxane and 100 mg of bis pinacolato diboron was added and the mixture was stirred at 100° C. for 2h. After completion of the reaction the mixture was concentrated and n-hexane (3 mL) added. The solid formed was filtered and submitted for purification by reverse phase prep HPLC to yield the desired product N-(4-((3-((difluoromethyl)sulfonyl)pyridin-2-yl)amino)-5-propionylpyridin-2-yl)cyclopropanecarboxamide as TFA salt (3.0 mg, 5.13 μmol, 1.006% yield).

HPLC-01: Method information

### Column: Kinetex Biphenyl (100×4.6) mm, 2.6μm

Mobile phase A: 0.05% TFA in water:ACN (95:5)
Mobile phase B: ACN:0.05% TFA in water (95:5)
Flow: 1.0 mL/min.

### RT=7.484, Purity=92.5%

HPLC-02: Method information

### Column: Kinetex EVO C18 (100×4.6) mm, 2.6μm

Mobile phase A: 0.05% TFA in water:ACN (95:5)
Mobile phase B: ACN:0.05% TFA in water (95:5)
Flow: 1.0 mL/min

### RT=6.136, Purity=92%

LCMS: Method information:

### Column: Kinetex XB-C18 (75×3.0) mm, 2.6μm

Mobile Phase A: 5 mM Ammonium formate pH 3.3:ACN (98:02)

### Mobile Phase B: ACN:Buffer (98:02)

Flow Rate: 1.0 ml/min.
Desired Product mass m/z=425.0 (M+1); Retention time=2.028 min.

### ¹H-NMR (400 MHz, MeOD): δ 9.03 (s, 1H), 8.91 (s, 1H), 8.81 (d, J=3.20 Hz, 1H), 8.45 (d, J=7.20 Hz, 1H), 7.45 (t, J=8.00 Hz, 1H), 6.96 (t, J=52.40 Hz, 2H), 3.16 (t, J=1.60 Hz, 3H), 1.91-1.93 (m, 1H), 1.24-1.34 (m, 3H), 1.03-1.22 (m, 4H),

### EXAMPLE 16

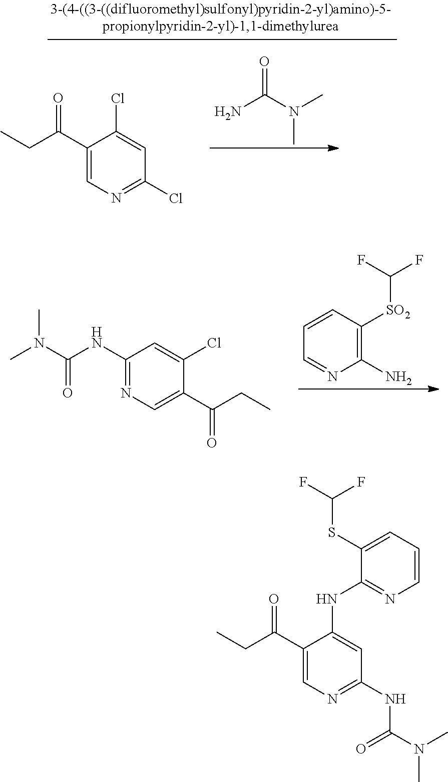

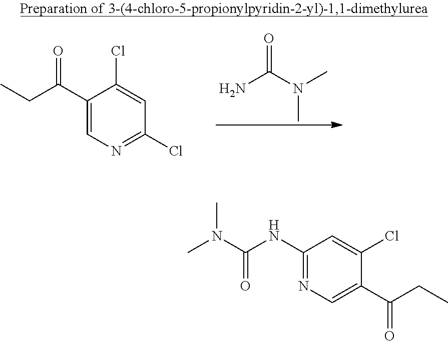

To a stirred solution of 1-(4,6-dichloropyridin-3-yl)propan-1-one (375 mg, 1.838 mmol) and 1,1-dimethylurea (243 mg, 2.76 mmol) in 1,4-dioxane (15 mL) was added Cs₂CO₃ (1796 mg, 5.51 mmol) at ambient temperature, then the reaction mixture was degassed for 5 min under N₂. Then, BINAP (114 mg, 0.184 mmol) and Pd₂(dba)₃ (84 mg, 0.092 mmol) were added to the reaction mixture and degassed for another 5 min. The resulting mixture was heated at 130° C. for 0.5 h in a sealed tube. After completion, the reaction mixture was diluted with ethyl acetate (100 mL), filtered through a celite pad and the celite washed with ethyl acetate (100 mL). The combined filtrate was washed with water (100 mL) followed by brine (100 mL), dried over anhydrous Na₂SO₄, filtered, and concentrated under reduced pressure. The crude residue was purified by column chromatography using silica gel (230:400), eluting with 0-50% ethyl acetate in petroleum ether to afford desired product as yellow solid.

MS (M+1) m/z: 256.0 (M+H)+. LC retention time 1.01 min [Method D].

The following Intermediates (3-8) were prepared in a similar manner to the preparation of intermediate 2.

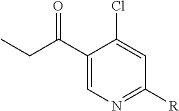

| | | | | Rt |
|---|---|---|---|---|
| Inter- | | | m/z | (min) |
| mediate | R | MW | [M + H]⁺ | Method |
| 17 | 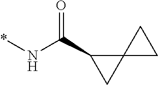 [SMILES: *NC(=O)[C@@H]1CC12CC2] | 278.74 | 279.0 | 2.26 [A] |
| 18 | 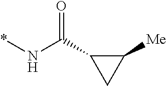 | 266.73 | 267.0 | 1.37 [D] |
| 19 | 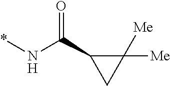 [SMILES: *NC(=O)[C@@H]1CC1(C)C] | 280.75 | 281.2 | 1.51 [D] |
| 20 | 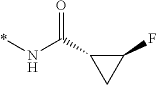 [SMILES: *NC(=O)[C@H]1C[C@@H]1F] | 270.69 | 271.0 | 1.26 [C] |
| 21 | 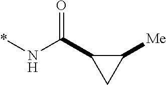 | 266.73 | 267.0 | 1.35 [C] |
| 22 | 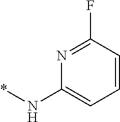 [SMILES: *Nc1cccc(F)n1] | 279.70 | 280.0 | 1.48 [D] |

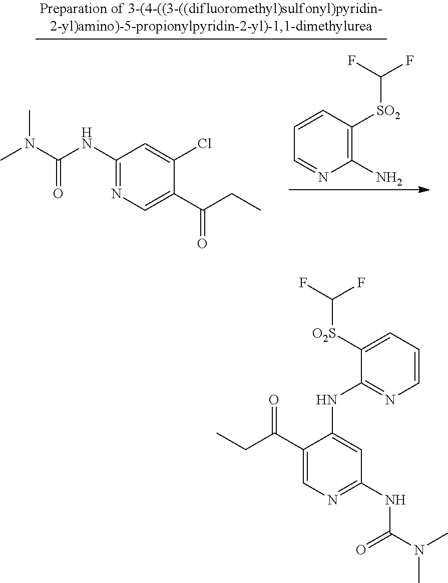 [SMILES: CCC(=O)c1cnc(NC(=O)N(C)C)cc1Cl ; Nc1ncccc1S(=O)(=O)C(F)F ; CCC(=O)c1cnc(NC(=O)N(C)C)cc1Nc1ncccc1S(=O)(=O)C(F)F]

To a stirred solution of 3-(4-chloro-5-propionylpyridin-2-yl)-1,1-dimethylurea (300 mg, 1.173 mmol) and 3-((difluoromethyl)sulfonyl)pyridin-2-amine (318 mg, 1.525 mmol) in 1,4-dioxane (15 mL) was added potassium phosphate (747 mg, 3.52 mmol) at ambient temperature, then the reaction mixture was degassed for 5 minutes under N₂. Then, Ruphos Pd G4 (200 mg, 0.235 mmol) was added to the reaction mixture and degassed for another 5 min. The resulting reaction mixture was heated at 130° C. for 1.5 h in a sealed tube. After completion, the reaction mixture was diluted with ethyl acetate (50 mL), filtered through celite pad and the pad washed with ethyl acetate (100 mL). The combined filtrate was washed with water (20 mL), followed by brine (20 mL), then dried over anhydrous Na₂SO₄ and concentrated under reduced pressure. The crude residue was purified by RP column chromatography and collected fractions were lyophilized to obtain desired 3-(4-((3-((difluoromethyl)sulfonyl)pyridin-2-yl)amino)-5-propionylpyridin-2-yl)-1,1-dimethylurea (41 mg, 0.087 mmol, 7.44% yield) as an off-white solid.

MS (M+1) m/z: 428.2 (M+H)+. LC retention time 1.68 min [Method A].

### ¹H-NMR (400 MHz, DMSO-d6): δ 11.98 (s, 1H), 9.15 (s, 1H), 8.88 (s, 1H), 8.74-8.72 (m, 2H), 8.33 (dd, J=1.60, 8.00 Hz, 1H), 7.51-7.25 (m, 2H), 3.10 (q, J=7.20 Hz, 2H), 2.93 (s, 6H), 1.09 (t, J=7.20 Hz, 3H).

The following examples were prepared in a similar manner to the preparation of example 16.

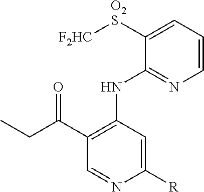

| | | | m/z | Rt (min) |
|---|---|---|---|---|
| Example | R | MW | [M + H]⁺ | Method] |
| 17 | 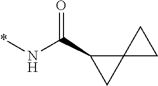 [SMILES: *NC(=O)[C@@H]1CC12CC2] | 450.46 | 451.1 | 2.35 [B] |
| 18 | 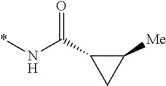 [SMILES: *NC(=O)[C@H]1C[C@@H]1C] | 438.45 | 439.2 | 2.60 [A] |
| 19 | 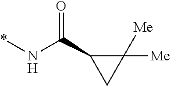 [SMILES: *NC(=O)[C@@H]1CC1(C)C] | 452.48 | 453.2 | 2.84 [A]ª |
| 20 | 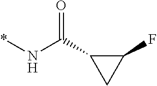 [SMILES: *NC(=O)[C@H]1C[C@@H]1F] | 442.41 | 443.0 | 2.11 [A]^{a} |
| 21a | 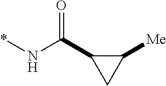 | 438.45 | 439.0 | 2.25 [A] ^{a,} * |
| 21b | 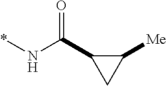 | 438.45 | 439.0 | 2.25 [A] ^{a,} * |
| 22 | 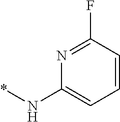 [SMILES: *Nc1cccc(F)n1] | 451.42 | 452.1 | 2.33 [B] |

‘a’ NaHCO₃ used in the Buchwald reaction instead of K₃PO₄.

‘*’ The cis isomers were separated from chiral SFC.

| Example No. | ¹H NMR |
|---|---|
| 16 | ¹H-NMR (400 MHz, DMSO-d₆): δ 12.03 (s, 1H), 10.83 (s, 1H), 9.06 (s, 1H), |
| | 8.96 (s, 1H), 8.75 (dd, J = 2.00, 4.60 Hz, 1H), 8.35 (dd, J = 1.60, 8.0 Hz, |
| | 1H), 7.52-7.26 (m, 2H), 3.13 (q, J = 7.20 Hz, 2H), 2.42-2.39 (m, |
| | 1H), 1.40-1.34 (m, 2H), 1.09 (t, J = 7.20 Hz, 3H), 0.91-0.76 (m, 4H). |
| 17 | ¹H-NMR (400 MHz, DMSO-d₆): δ 12.03 (s, 1H), 10.97 (s, 1H), 9.02 (s, 1H), |
| | 8.97 (s, 1H), 8.73 (dd, J = 2.00, 4.60 Hz, 1H), 8.35 (d, J = 2.00 Hz, 1H), |
| | 7.51-7.39 (m, 2H), 3.13 (q, J = 7.20 Hz, 2H), 1.82-1.80 (m, 1H), 1.25-1.23 |
| | (m, 1H), 1.10-1.01 (m, 7H), 0.70-0.68 (m, 1H). |
| 18 | ¹H-NMR (400 MHz, DMSO-d₆): δ 12.03 (s, 1H), 10.86 (s, 1H), 9.05 (s, 1H), |
| | 8.96 (s, 1H), 8.73 (dd, J = 1.60, 4.80 Hz, 1H), 8.34 (dd, J = 1.60, 7.80 Hz, |
| | 1H), 7.52-7.38 (m, 2H), 3.13 (q, J = 7.20 Hz, 2H), 1.94-1.92 (m, 1H), 1.12- |
| | 1.08 (m, 9H), 1.00-0.98 (m, 1H), 0.85-0.79 (m, 1H). |
| 19 | ¹H-NMR (400 MHz, DMSO-d₆): δ 12.04 (s, 1H), 11.18 (s, 1H), 9.00 (d, J = |
| | 12.40 Hz, 2H), 8.71 (d, J = 3.20 Hz, 1H), 8.34 (d, J = 7.60 Hz, 1H), 7.52- |
| | 7.26 (m, 2H), 4.89 (dd, J = 2.80, 65.00 Hz, 1H), 3.14 (q, J = 7.20 Hz, 2H), |
| | 2.53-2.60 (m, 1H), 1.57-1.51 (m, 1H), 1.26-1.21 (m, 1H), 1.10 (t, J = 7.20 |
| | Hz, 3H). |
| 20 | ¹H-NMR (400 MHz, DMSO-d₆): δ 12.03 (s, 1H), 10.93 (s, 1H), 9.05 (s, 1H), |
| | 8.96 (s, 1H), 8.72 (dd, J = 2.0, 4.6 Hz, 1H), 8.34 (dd, J = 2.0, 8.0 Hz, 1H), |
| | 7.52-7.26 (m, 2H), 3.15-3.09 (q, J = 7.2 Hz, 2H), 2.09-2.02 (m, 1H), 1.31- |
| | 1.22 (m, 1H), 1.15-1.11 (m, 6H), 1.09-0.95 (m, 1H), 0.85-0.75 (m, 1H). |
| 21 | ¹H-NMR (400 MHz, DMSO-d₆): δ 12.03 (s, 1H), 10.93 (s, 1H), 9.05 (s, 1H), |
| | 8.96 (s, 1H), 8.72 (dd, J = 2.0, 4.6 Hz, 1H), 8.34 (dd, J = 2.0, 8.0 Hz, 1H), |
| | 7.52-7.26 (m, 2H), 3.15-3.09 (q, J = 7.2 Hz, 2H), 2.09-2.02 (m, 1H), 1.31- |
| | 1.22 (m, 1H), 1.15-1.11 (m, 6H), 1.09-0.95 (m, 1H), 0.85-0.75 (m, 1H). |
| 22 | ¹H-NMR (400 MHz, DMSO-d₆): δ 12.18 (s, 1H), 10.43 (s, 1H), 8.97 (d, J = |
| | 7.2 Hz, 2H), 8.78 (dd, J = 2.0, 4.6 Hz, 1H), 8.36 (dd, J = 2.0, 8.0 Hz, 1H), |
| | 7.89 (dd, J = 8.0, 16.8 Hz, 1H), 7.62 (dd, J = 2.0, 8.0 Hz, 1H ), 7.54-7.28 |
| | (m, 2H), 6.68 (dd, J= 2.4, 7.6 Hz, 1H), 3.11 (q, J = 7.2 Hz, 2H), 1.12 (t, J = |
| | 7.2 Hz, 3H). |

### BIOLOGICAL ASSAYS

The following assays are used to show the activity for compounds of the invention.

### IFNα-Induced STAT Phosphorylation in Human Whole Blood

After an hour long incubation with compound, human whole blood (drawn with ACD-A as anticoagulant) was stimulated with 1000 U/mL recombinant human IFNα A/D (R&D Systems 11200-2) for 15 min. The stimulation was stopped by adding Fix/Lyse buffer (BD 558049). Cells were stained with a CD3 FITC antibody (BD 555916), washed, and permeabilized on ice using Perm III buffer (BD 558050). Cells were then stained with an Alexa-Fluor 647 pSTAT5 (pY694) antibody (BD 612599) for 60 min prior to analysis on the iQue Plus. The amount of pSTAT5 expression was quantitated by median fluorescence intensity after gating on the CD3 positive population.

TABLE 1
Potency of exemplified compounds in human whole
blood assay is shown below in Table 1.

| | Human Whole blood IFNa |
|---|---|
| Example No. | pSTAT5 IC₅₀ (μM) |
| 1 | 0.07 |
| 2 | 0.67 |
| 3 | 0.59 |
| 4 | 0.03 |
| 5 | 0.25 |
| 6 | 0.21 |
| 13a | 0.11 |
| 13b | 0.01 |
| 15 | 0.15 |
| 16 | 0.98 |
| 17 | 0.96 |
| 18 | 0.85 |
| 20 | 0.46 |
| 21a | 1.40 |
| 21b | 0.05 |
| 22 | 0.22 |

We claim:

1. A compound of formula I 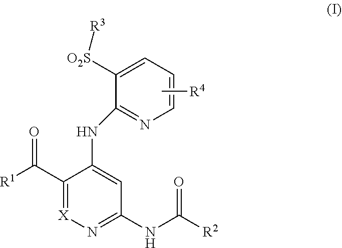
or a stereoisomer or pharmaceutically acceptable salt thereof, wherein
X is —N— or —CH—,
R¹ is C₁₋₃ alkyl or —NHCD₃;
R²is —N(CH₃)₂, —OR^{2a} or C₃₋₆ cycloalkyl substituted with 0-2 R^{2b};
R^{2a} is C₁₋₃ alkyl;
R^{2b} is F or C₁₋₃ alkyl;
R³ is C₁₋₃ fluoroalkyl or C₃₋₆ cycloalkyl; and
R⁴ is hydrogen, halogen or C₁₋₃ alkyl.

2. The compound according to claim 1 of the formula 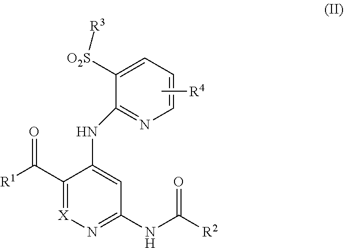
or a stereoisomer or pharmaceutically acceptable salt thereof, wherein
X is —N— or —CH—,
R¹ is C₁₋₃ alkyl or —NHCD₃;
R²is —N(CH₃)₂, —OR^{2a} or C₃₋₆ cycloalkyl substituted with 0-2 R^{2b};
R^{2a} is C₁₋₃ alkyl;
R^{2b} is F or C₁₋₃ alkyl;
R³ is CHF₂ or C₃₋₆ cycloalkyl; and
R⁴ is hydrogen, halogen or C₁₋₃ alkyl.

3. The compound according to claim 1 of formula II 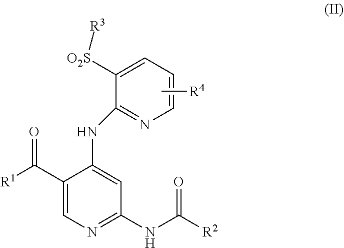
or a stereoisomer or pharmaceutically acceptable salt thereof, wherein
R¹ is C₁₋₃ alkyl or —NHCD₃;
R²is —N(CH₃)₂, —OR^{2a} or C₃₋₆ cycloalkyl substituted with 0-2 R^{2b};
R^{2a} is C₁₋₃ alkyl;
R^{2b} is F or C₁₋₃ alkyl;
R³ is CHF₂ or C₃₋₆ cycloalkyl; and
R⁴ is hydrogen, halogen or C₁₋₃ alkyl.

4. The compound according to claim 3 of the formula 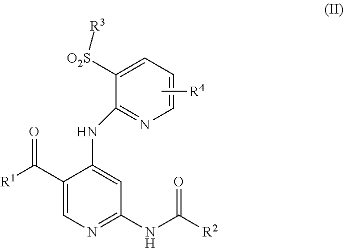
or a stereoisomer or pharmaceutically acceptable salt thereof, wherein
R¹ is C₁₋₃ alkyl;
R²is —N(CH₃)₂, —OR^{2a} or C₃₋₆ cycloalkyl substituted with 0-2 R^{2b};
R^{2a} is C₁₋₃ alkyl;
R^{2b} is F or C₁₋₃ alkyl;
R³ is CHF₂ or C₃₋₆ cycloalkyl; and
R⁴ is hydrogen, halogen or C₁₋₃ alkyl.

5. The compound according to claim 1 of formula III 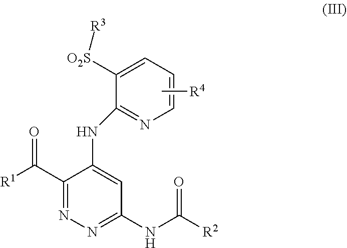
or a stereoisomer or pharmaceutically acceptable salt thereof, wherein
R¹ is C₁₋₃ alkyl or —NHCD₃;
R²is —N(CH₃)₂, —OR^{2a} or C₃₋₆ cycloalkyl substituted with 0-2 R^{2b};
R^{2a} is C₁₋₃ alkyl;
R^{2b} is F or C₁₋₃ alkyl;
R³ is CHF₂ or C₃₋₆ cycloalkyl; and
R⁴ is hydrogen, halogen or C₁₋₃ alkyl.

6. The compound according to claim 5 of the formula 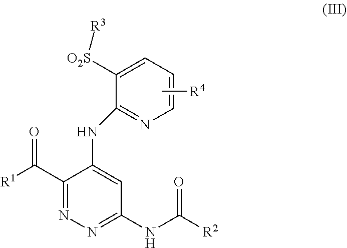
or a stereoisomer or pharmaceutically acceptable salt thereof, wherein
R¹ is —NHCD₃;
R²is —N(CH₃)₂, —OR^{2a} or C₃₋₆ cycloalkyl substituted with 0-2 R^{2b};
R^{2a} is C₁₋₃ alkyl;
R^{2b} is F or C₁₋₃ alkyl;
R³ is CHF₂ or C₃₋₆ cycloalkyl; and
R⁴ is hydrogen, halogen or C₁₋₃ alkyl.

7. A compound or a pharmaceutically acceptable salt thereof, selected from
6-(cyclopropanecarboxamido)-4-((3-(cyclopropylsulfonyl)-pyridin-2-yl)amino)-N-(methyl-d₃)pyridazine-3-carboxamide,
methyl (5-((3-(cyclopropyl-sulfonyl)pyridin-2-yl)amino)-6-((methyl-d₃)-carbamoyl)-pyridazin-3-yl)-carbamate,
N-(4-((3-(cyclopropyl-sulfonyl)pyridin-2-yl)amino)-5-propionylpyridin-2 -yl)cyclo-propanecarboxamide,
6-(cyclopropanecarboxamido)-4-((3-((difluoromethyl)-sulfonyl)pyridin-2-yl)amino)-N-(methyl-d₃)pyridazine-3-carboxamide,
methyl (5-((3-((difluoromethyl)sulfonyl)pyridin-2-yl)amino)-6-((methyl-d₃)carbamoyl)pyridazin-3-yl) carbamate,
4-((3-((difluoromethyl)sulfonyl)pyridin-2-yl)amino)-6-(3,3-dimethylureido)-N-(methyl-d₃)pyridazine-3-carboxamide,
4-((3-((difluoromethyl)sulfonyl)pyridin-2-yl)amino)-N-(methyl-d₃)-6-(3-methyl-2-oxoimidazolidin-1-yl)pyridazine-3-carboxamide,
4-((3-((difluoromethyl)sulfonyl)pyridin-2-yl)amino)-N-(methyl-d₃)-6-((1R,2R)-2-methylcyclopropane-1-carboxamido)pyridazine-3-carboxamide,
(R)-4-((3-((difluoromethyl)sulfonyl)pyridin-2-yl)amino)-N-(methyl-d₃)-6-(spiro[2.2]pentane-1-carboxamido)pyridazine-3-carboxamide,
4-((3-((difluoromethyl)sulfonyl)pyridin-2-yl)amino)-N-(methyl-d₃)-6-((1S,2S)-2-methylcyclopropane-1-carboxamido)pyridazine-3-carboxamide,
(R)-4-((3-((difluoromethyl)sulfonyl)pyridin-2-yl)amino)-6-(2,2-dimethylcyclopropane-1-carboxamido)-N-(methyl-d₃)pyridazine-3-carboxamide,
4-((3-((difluoromethyl)sulfonyl)pyridin-2-yl)amino)-6-((1R,2S)-2-fluorocyclopropane-1-carboxamido)-N-(methyl-d₃)pyridazine-3-carboxamide,
4-((3-((difluoromethyl)sulfonyl)pyridin-2-yl)amino)-N-(methyl-d₃)-6-(2-methylcyclopropane-1-carboxamido)pyridazine-3-carboxamide,
4-((3-((difluoromethyl)sulfonyl)pyridin-2-yl)amino)-6-((6-fluoropyridin-2-yl)amino)-N-(methyl-d₃)pyridazine-3-carboxamide,
N-(4-((3-((difluoromethyl)sulfonyl)pyridin-2-yl)amino)-5-propionylpyridin-2-yl)cyclopropanecarboxamide,
3-(4-((3-((difluoromethyl)sulfonyl)pyridin-2-yl)amino)-5-propionylpyridin-2-yl) -1,1-dimethylurea,
((R)-N-(4-((3-((difluoromethyl)sulfonyl)pyridin-2-yl)amino)-5-propionylpyridin-2-yl)spiro[2.2]pentane-1-carboxamide,
(1S,2S)-N-(4-((3-((difluoromethyl)sulfonyl)pyridin-2-yl)amino)-5-propionylpyridin-2-yl)-2-methylcyclopropane-1-carboxamide,
(R)-N-(4-((3-((difluoromethyl)sulfonyl)pyridin-2-yl)amino)-5-propionylpyridin-2-yl)-2,2-dimethylcyclopropane-1-carboxamide,
(1R,2S)-N-(4-((3-((difluoromethyl)sulfonyl)pyridin-2-yl)amino)-5-propionylpyridin-2-yl)-2-fluorocyclopropane-1-carboxamide,
N-(4-((3-((difluoromethyl)sulfonyl)pyridin-2-yl)amino)-5-propionylpyridin-2-yl)-2-methylcyclopropane-1-carboxamide, and
1-(4-((3-((difluoromethyl)sulfonyl)pyridin-2-yl)amino)-6-((6-fluoropyridin-2-yl)amino)pyridin-3-yl)propan-1-one.

8. A pharmaceutical composition comprising one or more compounds according to claim 1 and a pharmaceutically acceptable carrier or diluent.

9. A pharmaceutical composition comprising one or more compounds according to claim 4 and a pharmaceutically acceptable carrier or diluent.

10. A method of treating a disease, comprising administering to a patient in need of such treatment a therapeutically-effective amount of a compound according to claim 1, wherein the disease is a neurodegenerative disease.

11. The method of claim 10 wherein the neurodegenerative disease is Alzheimer's disease, Parkinson's disease, ALS or Multiple Sclerosis.
